(19) United States
(12) Patent Application Publication
(10) Pub. No.: US 2011/0217703 A1
(43) Pub. Date: Sep. 8, 2011

(54) P2/P2A/P2B GENE SEQUENCES AS DIAGNOSTIC TARGETS FOR THE IDENTIFICATION OF FUNGAL AND YEAST SPECIES

(75) Inventors: Thomas Gerard Barry, Kinarva (IE); Terry James Smith, Galway (IE); Marcin Jankiewicz, Galway (IE); Louise O'Connor, Galway (IE); Nina Tuite, Galway (IE); Sinead Lahiff, Gort (IE); Majella Maher, Moycullen (IE)

(73) Assignee: National University of Ireland Galway

(21) Appl. No.: 12/997,471

(22) PCT Filed: Jun. 15, 2009
(86) PCT No.: PCT/EP2009/057338

§ 371 Date: May 9, 2011

(30) Foreign Application Priority Data
Jun. 13, 2008 (IE) 2008/0489

(51) Int. Cl. C12Q 1/68; C07H 21/04

(52) U.S. Cl. 435/6.11; 536/24.32

(57) ABSTRACT

The present invention relates to nucleic acid primers and probes to detect one or more fungal and yeast species. More specifically the invention relates to the P2, P2A and P2B gene sequences (also known as 60S acidic ribosomal protein P2, RLA-2-ASPFU, Allergen ASP f8 or Afp2), the corresponding RNA, specific probes, primers and oligonucleotides related thereto and their use in diagnostic assays to detect and/or discriminate fungal and yeast species.

### FIELD OF THE INVENTION

The present invention relates to nucleic acid primers and probes to detect one or more fungal and yeast species. More specifically the invention relates to the P2, P2A and P2B gene sequences (also known as 60S acidic ribosomal protein P2, RLA-2-ASPFU, Allergen ASP f8 or Afp2), the corresponding RNA, specific probes, primers and oligonucleotides related thereto and their use in diagnostic assays to detect and/or discriminate fungal and yeast species.

### BACKGROUND TO THE INVENTION

Yeast and fungal infections represent a major cause of morbidity and mortality among immunocompromised patients. The number of immunocompromised patients at risk of yeast and fungal infection continues to increase each year, as does the spectrum of fungal and yeast agents causing disease. Mortality from fungal infections, particularly invasive fungal infections, is 30% or greater in certain risk groups. The array of available anti-fungal agents is growing; however, so too is the recognition of both intrinsic and emerging resistance to antifungal drugs. These factors are contributing to the increased need for cost containment in laboratory testing and have led to laboratory consolidation in testing procedures.

Invasive fungal infections are on the increase. In 2003, it was estimated that there were 9 million at risk patients of which 1.2 million developed infection. Candida spp. and Aspergillus spp. now rank as the most prominent pathogens infecting immunosupressed patients. In particular, infections are common in the urinary tract, the respiratory system and the bloodstream, at the site of insertion of stents, catheters and orthopaedic joints. Approximately, 10% of the known Candida spp. have been implicated in human infection. Invasive candidiasis occurs when candida enters the bloodstream and it is estimated to occur at a frequency of 8/100,000 population in the US with a mortality rate of 40%. Candida albicans is the 4^{th} most common cause of bloodstream infection. Aspergillosis usually begins as a pulmonary infection that can progress to a life-threatening invasive infection in some patients and has a mortality rate of greater than 90%. Emerging mycoses agents include Fusarium, Scedosporium, Zygomycetes and Trichosporon spp. (“Stakeholder Insight: Invasive fungal infections”, Datamonitor, January 2004).

Immunocompromised patients including transplant and surgical patients, neonates, cancer patients, diabetics and those with HIV/AIDs are at high risk of developing invasive fungal infections (Datamonitor report: Stakeholder opinion—Invasive fungal infections, options outweigh replacements 2004). A large number of severe cases of sepsis are reported each year. Despite improvements in its medical management, sepsis still constitutes one of the greatest challenges in intensive care medicine. Microorganisms (bacteria, fungi and yeast) responsible for causing sepsis are traditionally detected in hospital laboratories with the aid of microbiological culture methods with poor sensitivity (25-82%), which are very time-consuming, generally taking from two to five days to complete, and up to eight days for the diagnosis of fungal infections. Definitive diagnosis of an infection caused by a yeast or fungus is usually based on either, the recovery and identification of a specific agent from clinical specimens or microscopic demonstration of fungi with distinct morphological features. However, there are numerous cases where these methods fail to provide conclusive proof as to the infecting agent. In these instances, the detection of specific host antibody responses can be used, although again this can be affected by the immune status of the patient. Time is critical in the detection and identification of bloodstream infections typically caused by bacteria and fungi. Effective treatment depends on finding the source of infection and making appropriate decisions about antibiotics or antifungals quickly and efficiently. Only after pathogens are correctly identified can targeted therapy using a specific antibiotic or anti-fungal begin. Many physicians would like to see the development of better in vitro amplification and direct detection diagnostic techniques for the early diagnosis of yeast and fungi (“Stakeholder Insight: Invasive fungal infections”, Datamonitor, January 2004). Recently Roche™ launched a real time PCR based assay (Septifast™), for the detection of bacterial, fungal and yeast DNA in clinical samples. Therefore, there is a clear need for the development of novel rapid diagnostic tests for clinically significant bacterial and fungal pathogens for bioanalysis applications in the clinical sector. This has led the current inventors to identify novel fungal and yeast nucleic acid targets for application in Nucleic Acid Diagnostics (NAD) tests. Fungal and yeast nucleic acid based diagnostics have focused heavily on the ribosomal RNA (rRNA) genes, RNA transcripts, and their associated DNA/RNA regions. The rRNA genes are highly conserved in all fungal species and they also contain divergent and distinctive intergenic transcribed spacer regions. Ribosomal rRNA comprises three genes: the large sub-unit gene (28S), the small sub-unit gene (18S) and the 5.8S gene. The 28S and 18S rRNA genes are separated by the 5.8S rRNA and two internal transcribed spacers (ITS 1 and ITS2). Because the ITS region contains a high number of sequence polymorphisms, numerous researchers have concentrated their efforts on these as targets (Atkins and Clark, 2004). rRNA genes are also multicopy genes with >10 copies within the fungal genome.

A number of groups are working on developing new assays for fungal and yeast infections. U.S. 2004044193 relates to, amongst a number of other aspects, the transcription factor CaTEC1 of Candida albicans; inhibitors thereof, and methods for the diagnosis and therapy of diseases which are connected with a Candida infection; and also diagnostic and pharmaceutical compositions which contain the nucleotide sequences, proteins, host cells and/or antibodies. WO0183824 relates to hybridization assay probes and accessory oligonucleotides for detecting ribosomal nucleic acids from Candida albicans and/or Candida dubliniensis. U.S. Pat. No. 6,017,699 and U.S. Pat. No. 5,426,026 relate to a set of DNA primers, which can be used to amplify and speciate DNA from five medically important Candida species. U.S. Pat. No. 6,747,137 discloses sequences useful for diagnosis of Candida infections. EP 0422872 and U.S. Pat. No. 5,658,726 disclose probes based on 18S rRNA genes, and U.S. Pat. No. 5,958,693 discloses probes based on 28S rRNA, for diagnosis of a range of yeast and fungal species. U.S. Pat. No. 6,017,366 describes sequences based on chitin synthase gene for use in nucleic acid based diagnostics for a range of Candida species. It is clear though, that development of faster, more accurate diagnostic methods are required, particularly in light of the selection pressure caused by modern anti-microbial treatments which give rise to increased populations of resistant virulent strains with mutated genome sequences. Methods that enable early diagnosis of microbial causes of infection enable the selection of a specific narrow spectrum antibiotic or antifungal to treat the infection (Datamonitor report: Stakeholder opinion—Invasive fungal infections, options outweigh replacements 2004; Datamonitor report: Stakeholder Opinion—Sepsis, under reaction to an overreaction, 2006).

Ribosomes are a two part organelle, composed of a large and a small subunit. The large subunit possesses a number of features which are conserved in all organisms for example the presence of a complex of a number of acidic proteins, the P proteins which form a stalk-like structure (Abramczyk et al., 2004 Tchórzewski et al., 2000; 2003). In the 60S ribosomal subunit of eukaryotes these acidic P proteins include, P0, P1 and P2 (Wool et al 1991). The complex formed by these P proteins is required for efficient translation. In yeasts, there are two isoforms of P1 (P1A and P1B) and P2 (P2A and P2B) which are encoded by four distinct single copy genes (Newton et al, 1990, Bailey-Serres et al., 1997).

This invention relates to the use of the P2A, P2B genes in yeast and the P2 gene in fungi. There are currently 65 P2B gene sequences (approx. 360 bp) available in NCBI GenBank database including 3 P2B sequences for Candida albicans and one sequence of high homology for C. glabrata. There are 329 P2 sequences available in NCBI GenBank database including annotated sequences for two Aspergillus species and presumptive P2 sequences for three species. Additional P2B sequences of representative species of Candida and P2 sequences of representative species of Aspergillus were generated by the inventors following the design of PCR primers to amplify P2B gene regions in Candida and P2 gene regions in Aspergillus spp. The published and newly generated P2B and P2 gene sequences for Candida and Aspergillus spp. respectively, were aligned and analysed using bioinformatics tools. PCR primers and species-specific DNA probes for selected species, Candida glabrata and Aspergillus fumigatus were designed and demonstrated for molecular species identification using real-time PCR. These examples demonstrate the potential of P2A/P2B and P2 genes for molecular identification of species of Candida, Aspergillus and other yeast and fungal species.

### Definitions

“Synthetic oligonucleotide” refers to molecules of nucleic acid polymers of 2 or more nucleotide bases that are not derived directly from genomic DNA or live organisms. The term synthetic oligonucleotide is intended to encompass DNA, RNA, and DNA/RNA hybrid molecules that have been manufactured chemically, or synthesized enzymatically in vitro.

An “oligonucleotide” is a nucleotide polymer having two or more nucleotide subunits covalently joined together. Oligonucleotides are generally about 10 to about 100 nucleotides. The sugar groups of the nucleotide subunits may be ribose, deoxyribose, or modified derivatives thereof such as OMe. The nucleotide subunits may be joined by linkages such as phosphodiester linkages, modified linkages or by non-nucleotide moieties that do not prevent hybridization of the oligonucleotide to its complementary target nucleotide sequence. Modified linkages include those in which a standard phosphodiester linkage is replaced with a different linkage, such as a phosphorothioate linkage, a methylphosphonate linkage, or a neutral peptide linkage. Nitrogenous base analogs also may be components of oligonucleotides in accordance with the invention. A “target nucleic acid” is a nucleic acid comprising a target nucleic acid sequence. A “target nucleic acid sequence,” “target nucleotide sequence” or “target sequence” is a specific deoxyribonucleotide or ribonucleotide sequence that can be hybridized to a complementary oligonucleotide.

An “oligonucleotide probe” is an oligonucleotide having a nucleotide sequence sufficiently complementary to its target nucleic acid sequence to be able to form a detectable hybrid probe:target duplex under high stringency hybridization conditions. An oligonucleotide probe is an isolated chemical species and may include additional nucleotides outside of the targeted region as long as such nucleotides do not prevent hybridization under high stringency hybridization conditions. Non-complementary sequences, such as promoter sequences, restriction endonuclease recognition sites, or sequences that confer a desired secondary or tertiary structure such as a catalytic active site can be used to facilitate detection using the invented probes. An oligonucleotide probe optionally may be labelled with a detectable moiety such as a radioisotope, a fluorescent moiety, a chemiluminescent, a nanoparticle moiety, an enzyme or a ligand, which can be used to detect or confirm probe hybridization to its target sequence. Oligonucleotide probes are preferred to be in the size range of from about 10 to about 100 nucleotides in length, although it is possible for probes to be as much as and above about 500 nucleotides in length, or below 10 nucleotides in length.

A “hybrid” or a “duplex” is a complex formed between two single-stranded nucleic acid sequences by Watson-Crick base pairings or non-canonical base pairings between the complementary bases. “Hybridization” is the process by which two complementary strands of nucleic acid combine to form a double-stranded structure (“hybrid” or “duplex”). A “fungus” or “yeast” is meant any organism of the kingdom Fungi, and preferably, is directed towards any organism of the phylum Ascomycota. “Complementarity” is a property conferred by the base sequence of a single strand of DNA or RNA which may form a hybrid or double-stranded DNA:DNA, RNA:RNA or DNA:RNA through hydrogen bonding between Watson-Crick base pairs on the respective strands. Adenine (A) ordinarily complements thymine (T) or uracil (U), while guanine (G) ordinarily complements cytosine (C).

The term “stringency” is used to describe the temperature, ionic strength and solvent composition existing during hybridization and the subsequent processing steps. Those skilled in the art will recognize that “stringency” conditions may be altered by varying those parameters either individually or together. Under high stringency conditions only highly complementary nucleic acid hybrids will form; hybrids without a sufficient degree of complementarity will not form. Accordingly, the stringency of the assay conditions determines the amount of complementarity needed between two nucleic acid strands forming a hybrid. Stringency conditions are chosen to maximize the difference in stability between the hybrid formed with the target and the non-target nucleic acid. With “high stringency” conditions, nucleic acid base pairing will occur only between nucleic acid fragments that have a high frequency of complementary base sequences (for example, hybridization under “high stringency” conditions, may occur between homologs with about 85-100% identity, preferably about 70-100% identity). With medium stringency conditions, nucleic acid base pairing will occur between nucleic acids with an intermediate frequency of complementary base sequences (for example, hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

‘High stringency’ conditions are those equivalent to binding or hybridization at 42° C. in a solution consisting of 5×SSPE (43.8g/l NaCl, 6.9 g/l NaH₂PO₄H₂O and 1.85 g/l EDTA, ph adjusted to 7.4 with NaOH), 0.5% SDS, 5× Denhardt's reagent and 100 μg/l denatured salmon sperm DNA followed by washing in a solution comprising 0.1×SSPE, 1.0% SDS at 42° C. when a probe of about 500 nucleotides in length is used.

‘Medium stringency’ conditions are those equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5×Denhardt's reagent and 100 μg/ml denatured salmon sperm DNA followed by washing in a solution comprising 1.0×SSPE, 1.0% SDS at 42° C., when a probe of about 500 nucleotides in length is used.

‘Low stringency’ conditions are those equivalent to binding or hybridization at 42° C. in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5×Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C., when a probe of about 500 nucleotides in length is used.

In the context of nucleic acid in-vitro amplification based technologies, “stringency” is achieved by applying temperature conditions and ionic buffer conditions that are particular to that in-vitro amplification technology. For example, in the context of PCR and real-time PCR, “stringency” is achieved by applying specific temperatures and ionic buffer strength for hybridisation of the oligonucleotide primers and, with regards to real-time PCR hybridisation of the probe/s, to the target nucleic acid for in-vitro amplification of the target nucleic acid.

One skilled in the art will understand that substantially corresponding probes of the invention can vary from the referred-to sequence and still hybridize to the same target nucleic acid sequence. This variation from the nucleic acid may be stated in terms of a percentage of identical bases within the sequence or the percentage of perfectly complementary bases between the probe and its target sequence. Probes of the present invention substantially correspond to a nucleic acid sequence if these percentages are from about 100% to about 80% or from 0 base mismatches in about 10 nucleotide target sequence to about 2 bases mismatched in an about 10 nucleotide target sequence. In preferred embodiments, the percentage is from about 100% to about 85%. In more preferred embodiments, this percentage is from about 90% to about 100%; in other preferred embodiments, this percentage is from about 95% to about 100%

By “sufficiently complementary” or “substantially complementary” is meant nucleic acids having a sufficient amount of contiguous complementary nucleotides to form, under high stringency hybridization conditions, a hybrid that is stable for detection. The terms “identical” or percent “identity,” in the context of two or more nucleic acids or polypeptide sequences, refer to two or more sequences or subsequences that are the same or have a specified percentage of amino acid residues or nucleotides that are the same (i.e., 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm.nih.gov/BLAST/or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1987-2005, Wiley Interscience)). A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

“Nucleic acid” refers to deoxyribonucleotides or ribonucleotides and polymers thereof in either single- or double-stranded form, and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

By “nucleic acid hybrid” or “probe:target duplex” is meant a structure that is a double-stranded, hydrogen-bonded structure, preferably about 10 to about 100 nucleotides in length, more preferably 14 to 50 nucleotides in length, although this will depend to an extent on the overall length of the oligonucleotide probe. The structure is sufficiently stable to be detected by means such as chemiluminescent or fluorescent light detection, autoradiography, electrochemical analysis or gel electrophoresis. Such hybrids include RNA:RNA, RNA:DNA, or DNA:DNA duplex molecules.

“RNA and DNA equivalents” refer to RNA and DNA molecules having the same complementary base pair hybridization properties. RNA and DNA equivalents have different sugar groups (i.e., ribose versus deoxyribose), and may differ by the presence of uracil in RNA and thymine in DNA. The difference between RNA and DNA equivalents do not contribute to differences in substantially corresponding nucleic acid sequences because the equivalents have the same degree of complementarity to a particular sequence.

By “preferentially hybridize” is meant that under high stringency hybridization conditions oligonucleotide probes can hybridize their target nucleic acids to form stable probe:target hybrids (thereby indicating the presence of the target nucleic acids) without forming stable probe:non-target hybrids (that would indicate the presence of non-target nucleic acids from other organisms). Thus, the probe hybridizes to target nucleic acid to a sufficiently greater extent than to non-target nucleic acid to enable one skilled in the art to accurately detect the presence of (for example Candida) and distinguish these species from other organisms. Preferential hybridization can be measured using techniques known in the art and described herein.

By “theranostics” is meant the use of diagnostic testing to diagnose the disease, choose the correct treatment regime and monitor the patient response to therapy. The theranostics of the invention may be based on the use of an NAD assay of this invention on samples, swabs or specimens collected from the patient.

### OBJECT OF THE INVENTION

It is an object of the current invention to provide sequences and/or diagnostic assays to detect and identify one or more yeast and fungal species. The current inventors have made use of the P2, P2A and P2B gene sequences to design primers that are specific to Candida P2B genes and to Aspergillus P2 genes. Such primers not only allow the detection of yeast and fungal species but also allow distinction between Candida and Aspergillus spp. This has an advantage over the prior art in that if one wants to identify a fungal pathogen in a sample, which contains Candida as a commensal, the approach of using universal primers may not be successful. There is a strong possibility that the Candida will out-compete the fungal pathogen in the amplification process and will be preferentially amplified, resulting in failure to detect the disease-causing pathogen. The current invention further provides for primers and probes that allow discrimination between different Candida species and among different Aspergillus species.

### SUMMARY OF THE INVENTION

The present invention provides for a diagnostic kit for detection and identification of yeast and/or fungal species, comprising an oligonucleotide probe capable of binding to at least a portion of the P2, P2A or P2B gene or its corresponding mRNA. The oligonucleotide probe may have a sequence substantially homologous to or substantially complementary to a portion of the P2, P2A or P2B gene or its corresponding mRNA. It will thus be capable of binding or hybridizing with a complementary DNA or RNA molecule. The P2 gene may be a fungal P2 gene. The P2A gene may be a yeast gene. The P2B gene may be yeast P2B gene. The nucleic acid molecule may be synthetic. The oligonucleotide probe may have a sequence of SEQ ID NO 9, 10, 91-94, 105-109, or a sequence substantially homologous to or substantially complementary to those sequences, which can also act as a probe for the P2, P2A or P2B genes.

The kit may comprise more than one such probe. In particular the kit may comprise a plurality of such probes. In addition, the kit may comprise additional probes for other organisms, such as, for example, bacterial species or viruses.

The identified sequences are suitable not only for in vitro DNA/RNA amplification based detection systems but also for signal amplification based detection systems. Furthermore, the sequences of the invention identified as suitable targets provide the advantages of having significant intragenic sequence heterogeneity in some regions, which is advantageous and enables aspects of the invention to be directed towards group or species-specific targets, and also having significant sequence homogeneity in some regions, which enables aspects of the invention to be directed towards genus-specific yeast and fungal primers and probes for use in direct nucleic acid detection technologies, signal amplification nucleic acid detection technologies, and nucleic acid in vitro amplification technologies for yeast and fungal diagnostics. The P2, P2A and P2B sequences allow for multi-test capability and automation in diagnostic assays. One of the advantages of the sequences of the present invention is that the intragenic P2, P2A and P2B nucleotide sequences diversity between closely related yeast or fungal species enables specific primers and probes for use in diagnostics assays for the detection of yeast and fungi to be designed. The P2, P2A and P2B nucleotide sequences, both DNA and RNA can be used with direct detection, signal amplification detection and in vitro amplification technologies in diagnostics assays. The P2, P2A and P2B sequences allow for multi-test capability and automation in diagnostic assays.

The kit may further comprise a primer for amplification of at least a portion of the P2 and/or P2A and/or P2B genes. Suitably, the kit comprises a forward and a reverse primer for a portion of the P2, P2A and/or P2B gene.

The portion of the P2A gene may be equivalent to a portion of the region of the gene from by position 114 to by position 439 of C. albicans (AF317661.1).

The portion of the P2B gene may be equivalent to a portion of the region of the gene from base pair position 1 to position 335 in C. albicans (XM_{—}718047.1, XM_{—}717900.1) or position 8 to base pair position 359 of the P2B gene in C. albicans (AF317662.1). C. albicans.

Particularly preferred, are kits comprising, a probe for a portion of the P2A C. albicans gene and/or a probe for a portion of the region of the gene equivalent to base pair position 114 to by position 439 of C. albicans (AF317661.1).

Particularly preferred, are kits comprising, a probe for a portion of the P2B C. albicans gene and/or a probe for a portion of the region of the gene equivalent to base pair position 1 to position 359. Also preferred are probes for a portion of the region of the gene equivalent to base pair position 1 to position 335 in C. albicans (XM_{—}718047.1, XM_717900.1) or position 8 to by position 359 of the P2B gene in C. albicans (AF317662.1). Equivalent base pair positioning may be found in other organisms, but not necessarily in the same position.

The portion of the P2B gene may be equivalent to a portion of the region of the gene from base pair 24 to by position 158 in C. glabrata (XM_{—}444905.1). Particularly preferred, are kits comprising, a probe for a portion of the P2B C. glabrata gene and/or a probe for a portion of the region of the gene equivalent to base pair position 24 to base pair position 158 in C. glabrata. Sequences equivalent to base pair position 24 to base pair position 158 can be found in other organisms, but not necessarily in the same position. The kit may also comprise additional primers or probes.

The portion of the P2 gene may be equivalent to a portion of the region of the gene from base pair positions 1 to 326 in A. fumigatus. Particularly preferred, are kits comprising, a probe for a portion of the P2 A. fumigatus gene and/or a probe for a portion of the region of the gene equivalent to base pair position 1 to base pair position 326 in A. fumigatus. Equivalent sequences to base pair position 1 to base pair position 326 can be found in other organisms, but not necessarily in the same position. The kit may also comprise additional primers or probes.

The primer may have a sequence selected from the group SEQ ID NO 1-8, 85-90, 95-104, and a sequence substantially homologous to or substantially complementary to those sequences, which can also act as a primer for the P2, P2A and P2B genes. The primers may also be a primer which preferentially hybridizes to the same nucleotide sequence as is preferentially hybridized by the primers SEQ ID NO 1-8, 85-90, 95-104.

The kit may comprise at least one forward in vitro amplification primer and at least one reverse in vitro amplification primer, the forward amplification primer having a sequence selected from the group consisting of SEQ ID NO 1, 3, 7; 85-88, 95-99, and a sequence being substantially homologous or complementary thereto which can also act as a forward amplification primer for the P2A, P2B or P2 genes, and the reverse amplification primer having a sequence selected from the group consisting of SEQ ID NO 2, 4, 5, 6, 8, 89, 90, 100-104, and a sequence being substantially homologous or complementary thereto which can also act as a reverse amplification primer for the P2A, P2B or P2 genes

The diagnostic kit may be based on direct nucleic acid detection technologies, signal amplification nucleic acid detection technologies, and nucleic acid in vitro amplification technologies is selected from one or more of Polymerase Chain Reaction (PCR), Ligase Chain Reaction (LCR), Nucleic Acids Sequence Based Amplification (NASBA), Strand Displacement Amplification (SDA), Transcription Mediated Amplification (TMA), Branched DNA technology (bDNA) and Rolling Circle Amplification Technology (RCAT)), or other in vitro enzymatic amplification technologies.

The invention also provides a nucleic acid molecule selected from the group consisting of SEQ ID NO.1 to SEQ ID NO. 110 and sequences substantially homologous thereto, or substantially complementary to a portion thereof and having a function in diagnostics based on the P2 and/or P2A and P2B genes. The nucleic acid molecule may comprise an oligonucleotide having a sequence substantially homologous to or substantially complementary to a portion of a nucleic acid molecule of SEQ ID NO.1 to SEQ ID NO. 110. The invention also provides a method of detecting a target organism in a test sample comprising the steps of:

(i) Mixing the test sample with at least one oligonucleotide probe as defined above under appropriate conditions; and

(ii) hybridizing under high stringency conditions any nucleic acid that may be present in the test sample with the oligonucleotide to form a probe:target duplex; and

(iii) determining whether a probe:target duplex is present; the presence of the duplex positively identifying the presence of the target organism in the test sample.

The nucleic acid molecule and kits of the present invention may be used in a diagnostic assay to detect the presence of one or more yeast and/or fungal species, to measure yeast and/or fungal titres in a patient or in a method of assessing the efficacy of a treatment regime designed to reduce yeast and/or fungal titre in a patient or to measure yeast and/or fungal contamination in an environment. The environment may be a hospital, or it may be a food sample, an environmental sample e.g. water, an industrial sample such as an in-process sample or an end product requiring bioburden or quality assessment. The kits and the nucleic acid molecule of the invention may be used in the identification and/or characterization of one or more disruptive agents that can be used to disrupt the P2A, P2B or P2 gene function. The disruptive agent may be selected from the group consisting of antisense RNA, PNA, and siRNA.

In some embodiments of the invention, a nucleic acid molecule comprising a species-specific probe can be used to discriminate between species of the same genus. The oligonucleotides of the invention may be provided in a composition for detecting the nucleic acids of yeast and fungal target organisms. Such a composition may also comprise buffers, enzymes, detergents, salts and so on, as appropriate to the intended use of the compositions. It is also envisioned that the compositions, kits and methods of the invention, while described herein as comprising at least one synthetic oligonucleotide, may also comprise natural oligonucleotides with substantially the same sequences as the synthetic nucleotide fragments in place of, or alongside synthetic oligonucleotides.

The invention also provides for an in vitro amplification diagnostic kit for a target yeast and/or fungal organism comprising at least one forward in vitro amplification primer and at least one reverse in vitro amplification primer, the forward amplification primer being selected from the group consisting of one or more of a sequence being substantially homologous or complementary thereto which can also act as a forward amplification primer, and the reverse amplification primer being selected from the group consisting of one or more of or a sequence being substantially homologous or complementary thereto which can also act as a reverse amplification primer.

The invention also provides for a diagnostic kit for detecting the presence of candidate yeast and/or fungal species, comprising one or more DNA probes comprising a sequence substantially complementary to, or substantially homologous to the sequence of the P2A, P2B or P2 gene of the candidate yeast and/or fungal species. The present invention also provides for one or more synthetic oligonucleotides having a nucleotide sequence substantially homologous to or substantially complementary to one or more of the group consisting of the P2A, P2B gene or mRNA transcripts thereof, the yeast and or fungal P2 gene or mRNA transcript thereof, the yeast P2B gene or mRNA transcript thereof, one or more of SEQ ID NO 1-SEQ ID NO 110.

The nucleotide may comprise DNA. The nucleotide may comprise RNA. The nucleotide may comprise a mixture of DNA, RNA and PNA. The nucleotide may comprise synthetic nucleotides. The sequences of the invention (and the sequences relating to the methods, kits compositions and assays of the invention) may be selected to be substantially homologous to a portion of the coding region of the P2, P2A or P2B genes. The gene may be a gene from a target yeast or fungal organism. The sequences of the invention are preferably sufficient so as to be able form a probe:target duplex to the portion of the sequence.

The invention also provides for a diagnostic kit for a target yeast or fungal organism comprising an oligonucleotide probe substantially homologous to or substantially complementary to an oligonucleotide of the invention (which may be synthetic). It will be appreciated that sequences suitable for use as in vitro amplification primers may also be suitable for use as oligonucleotide probes: while it is preferable that amplification primers may have a complementary portion of between about 15 nucleotides and about 30 nucleotides (more preferably about 15-about 23, most preferably about 20 to about 23), oligonucleotide probes of the invention may be any suitable length. The skilled person will appreciate that different hybridization and or annealing conditions will be required depending on the length, nature & structure (eg. Hybridization probe pairs for LightCycler, Taqman 5′ exonuclease probes, hairpin loop structures etc. and sequence of the oligonucleotide probe selected.

Kits and assays of the invention may also be provided wherein the oligonucleotide probe is immobilized on a surface. Such a surface may be a bead, a membrane, a column, dipstick, a nanoparticle, the interior surface of a reaction chamber such as the well of a diagnostic plate or inside of a reaction tube, capillary or vessel or the like.

The target yeast or fungal organism may be selected from the group consisting of C. albicans, C. glabrata, C. krusei, C. parapsilosis, C. tropicalis C. dubliniensis, C. guilliermondii, C. norvegiensis, C. famata, C. haemuloni, C. kefyr, C. utilis, C. viswanathii, A. fumigatus, A. nidulans, A. clavatus, A. niger, A. terreus, A. flavus, A. versicolor and Neosartorya fischeri.

Under these circumstances, the amplification primers and oligonucleotide probes of the invention may be designed to a gene specific or genus specific region so as to be able to identify one or more, or most, or substantially all of the desired organisms of the target organism grouping.

The test sample may comprise cells of the target yeast and/or fungal organism. The method may also comprise a step for releasing nucleic acid from any cells of the target yeast or fungal organism that may be present in said test sample. Ideally, the test sample is a lysate of an obtained sample from a patient (such as a swab, or blood, urine, saliva, a bronchial lavage, dental specimen, skin specimen, scalp specimen, transplant organ biopsy, stool, mucus, or discharge sample). The test samples may be a food sample, a water sample, an environmental sample, an end product, end product or in-process industrial sample.

The invention also provides for the use of any one of SEQ ID NO.1 to SEQ ID NO.110 in a diagnostic assay for the presence of one or more yeast or fungal species. The species may be selected from the group consisting of C. albicans, C. glabrata, C. krusei, C. parapsilosis, C. tropicalis, C. dubliniensis, C. guilliermondii, C. norvegiensis, C. famata, C. haemuloni, C. kefyr, C. utilis, C. viswanathii, A. fumigatus, N. fischeri, A. clavatus, A. niger, A. terreus, A. flavus, A. versicolor and A. nidulans.

The invention also provides for kits for use in clinical diagnostics, theranostics, food safety diagnostics, industrial microbiology diagnostics, environmental monitoring, veterinary diagnostics, bio-terrorism diagnostics comprising one or more of the synthetic oligonucleotides of the invention. The kits may also comprise one or more articles selected from the group consisting of appropriate sample collecting instruments, reagent containers, buffers, labelling moieties, solutions, detergents and supplementary solutions. The invention also provides for use of the sequences, compositions, nucleotide fragments, assays, and kits of the invention in theranostics, Food safety diagnostics, Industrial microbiology diagnostics, Environmental monitoring, Veterinary diagnostics, Bio-terrorism diagnostics.

The nucleic acid molecules, composition, kits or methods may be used in a diagnostic nucleic acid based assay for the detection of yeast and/or fungal species.

The nucleic acid molecules, composition, kits or methods may be used in a diagnostic assay to measure yeast and/or fungal titres in a patient. The titres may be measured in vitro.

The nucleic acid molecules, composition, kits or methods may be used in a method of assessing the efficacy of a treatment regime designed to reduce yeast and/or fungal titre in a patient comprising assessing the yeast and/or fungal titre in the patient (by in vivo methods or in vitro methods) at one or more key stages of the treatment regime. Suitable key stages may include before treatment, during treatment and after treatment. The treatment regime may comprise an antifungal agent, such as a pharmaceutical drug. The nucleic acid molecules, composition, kits or methods may be used in a diagnostic assay to measure potential yeast and/or fungal contamination, for example, in a hospital. The nucleic acid molecules, composition, kits or methods may be used in the identification and/or characterization of one or more disruptive agents that can be used to disrupt the P2, P2A or P2B gene function. Suitable disruptive agents may be selected from the group consisting of antisense RNA, PNA, siRNA.

### BRIEF DESCRIPTION OF THE DRAWINGS

FIG. 1: Primers binding sites (grey highlights or bold text or bold and underlined) in P2B of Candida albicans.

FIG. 2: Primers binding sites in P2 of Aspergillus fumigatus. The amplified region of interest is underlined (Position 1 to 326).

FIG. 3: Binding site of C. glabrata probe P1-CglabP2B (bold and underlined) in the amplified fragment of P2B. PCR primers CglabP2B-F/CglabP2B-R are highlighted.

FIG. 4: Binding site of the A. fumigatus probe P1-AspP2 (bold and underlined) in the amplified fragment of P2B. PCR primers AspP2-F/AspP2-R are highlighted.

FIG. 5: Resulting amplification curve from Real-Time PCR assay based on the P2B gene for C. glabrata with P1-CglabP2B probe. Specificity of the assay was tested using a panel of DNA from 4 other Candida species and Aspergillus fumigatus. The 3 C. glabrata strains tested were detected and no cross-reaction was observed with DNA from other species.

FIG. 6. Resulting amplification curve from Real-time PCR based on the P2 gene for A. fumigatus with the P1-AspP2 probe. Specificity of the assay was tested against a panel of DNA from 6 closely related Aspergillus species and C. albicans. The assay detected A. fumigatus but did not detect or cross-react with DNA from C. albicans or significantly with any of the Aspergillus species tested.

FIG. 7. Alignment of sequence information for Aspergillus species.

### DETAILED DESCRIPTION OF THE INVENTION

### Materials and Methods

### Cell Culture

Candida species were cultured in Sabouraud broth (4% wt/vol glucose, 1% wt/vol peptone, 1.5% agar) for 48 hours at 37° C. in a shaking incubator. Aspergillus species were cultured in Sabouraud broth (4% wt/vol glucose, 1% wt/vol peptone, 1.5% agar) or agar for 3-4 days at 25° C.

### DNA Extraction

Cells from Candida and Aspergillus spp. were pretreated with lyticase or zymolase enzymes prior to DNA isolation. DNA was isolated Candida and Apergillus spp. using the MagNA Pure System (Roche Molecular Systems) in combination with the MagNA pure Yeast and Bacterial isolation kit III.

DNA sequencing of P2B/P2 gene regions in Candida and Aspergillus spp.

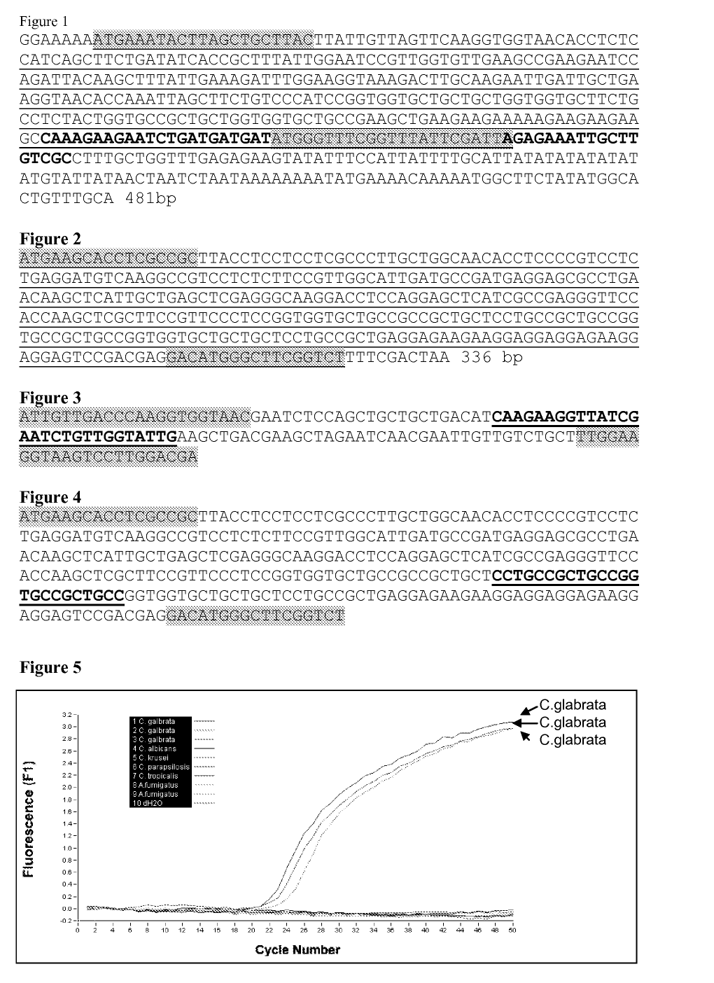

The available sequences of the P2B genes of Candida and P2 genes of Aspergillus spp. were acquired from the NCBI database and aligned using Clustal W. Three annotated sequences for P2B of C. albicans (XM_{—}718047.1, XM_{—}717900.1 and AF317662.1) are available in the NCBI database and a sequence of high homology to P2B in C. albicans has been deposited for Candida glabrata (hypothetical protein, XM_{—}444905.1), which is considered here to be P2B. In addition to available sequences for P2 in Aspergillus spp., three presumptive P2 sequences are deposited as hypothetical proteins for Aspergillus spp. (XM_{—}001213622.1, XM_{—}001397764.1, XM_{—}658508.1). PCR Primers were designed (Table 1) and synthesized by an external company, TibMolBiol, Germany. Primer set CanP2B-F/CanP2B-R was designed to amplify a 335 by region of the P2B in Candida spp. from positions equivalent to by position 1 to position 335 in C. albicans (XM_{—}718047.1, XM_{—}717900.1) or position 8 to by position 342 of the P2B gene in C. albicans (AF317662.1). Primer set CanP2B-F/CanP2B-R may also in selected Candida species amplify a 326 by region of the P2A gene. Primer set CanP2B-F/CanP2B flR was designed to amplify a 352 by region of the P2B equivalent to by 8 to by 359 in C. albicans (AF317662.1). Primer set P2B-F/P2BinR1 were designed to amplify a 312 by region of the P2B gene from by position 1 to by position 312 in XM718047.1/XM 717900.1 and from by position 8 to by 319 of AF317662.1. Primer set CglabP2B-F/CglabP2B-R were designed to amplify the region in P2B equivalent to by position 24 to by position 158 in C. glabrata (XM_{—}444905.1). Primer set AspP2-F/AspP2-R were designed to amplify a region of P2 in Aspergillus spp. from base pair positions equivalent to 1 to position 326 in A. fumigatus (XM_{—}750250.1). FIGS. 1 and 2 show the positions of the primer set CanP2B-F/CanP2B-R,CanP2B flR and P2BinR1 in C. albicans and AspP2-F/AspP2-R in A. fumigatus respectively. These primer sets were used to amplify regions of the P2B and P2 genes in other Candida and Aspergillus spp. respectively by conventional PCR on the iCycler BioRad PCR machine or the PTC200 Peltier thermocycler (MJ Research) using the reagents outlined in Table 2 and the thermocycling conditions described in Table 3 or modifications thereof. The PCR reaction products purified with Roche High Pure PCR Product Purification kit or with the ExoSAP-IT kit (USB) according to the manufacturers' instructions were sent for sequencing to Sequiserve, Germany and were sequenced using the forward amplification primer CanP2B-F or AspP2-F. Sequence information was obtained for P2B gene regions for 8 Candida spp. (C. albicans, C. dubliniensis, C. glabrata, C. krusei, C. parapsilosis, C. tropicalis, C. guillermondii, C. lusitanie) and P2 gene regions for 7 Aspergillus species (A. fumigatus, A. nidulans, A. clavatus, A. niger, A. terreus, A. flavus, A. versicolor) and Neosartorya fischeri.

TABLE 1
PCR primers designed for amplification of P2B
gene region in Candida and P2 gene region in
Aspergillus spp.

| Primer Name | Primer Sequence 5′ to 3′ |
|---|---|
| CglabP2B-F | ATTGTTGACCCAAGGTGGTAAC |
| CglabP2B-R | TCGTCCAAGGACTTACCTTCCAA |
| CanP2B-F | ATGAAATACTTAGCTGCTTAC |
| CanP2B-R | TAATCGAATAAACCGAAACCCA |
| CanP2B-flR | GCGACAAGCAATTTCTCT |
| CanP2BinR1 | ATCATCATCAGATTCTTCTTTG |
| AspP2-F | ATGAAGCACCTCGCCGC |
| AspP2-R | AGACCGAAGCCCATGTC |
| P2ForA | GAGGAGCGCCT |
| P2ForB | GAGGAGCGCCTC |
| P2ForC | GGAGGAGCGCCTC |
| P2ForD | TGAGGAGCGCCTC |
| P2RevA | CCGGAGGGAACGGA |
| P2RevB | CCGGAGGGAACGG |
| CAP2BF (For) | ACCTCTCCATCAGCTTCTG |
| CAP2BR (Rev) | TCAGCAATCAATTCTTGC |
| CGP2BF (For) | ACCTCTGTCTTATCATCTGTCG |
| CKP2BR (Rev) | CTCTTCGACGGACTTACC |
| CGP2BF (For) | AAGAAGGTTATCGAATCTGTTG |
| CGP2BR (Rev) | TTCGTCCAAGGACTTACC |
| CTP2BF (For) | TCCGCTTTATTGGAACAAGTTG |
| CTP2BR (Rev) | TTCTTGCAAGTCTTTACC |
| CPP2BF (For) | TCCTCATTGTTGGAATCCGTTG |
| CPP2BR (Rev) | CTCGTTGATGTCTTTACC |

TABLE 2
PCR reagents used to amplify P2B gene region in Candida
and P2 gene region in Aspergillus spp.

| | SAMPLE | |
|---|---|---|
| PCR Reaction Mix | x1 | |
| 10x Buffer (100 mM Tris HCl, 15 mM MgCl₂, | 5 | μl |
| 500 mM KCl pH 8.3) | | |
| dNTP's Mix, Roche (10 mM dNTP) | 1 | μl |
| Primer Forward CanP2B-F or AspP2B-F (10 μM) | 1 | μl |
| Primer Reverse CanP2B-R CanP2B-flR, | 1 | μl |
| Can P2BinR or AspP2B-R (10 μM) | | |
| Polymerase TaqPol, Roche 1 U/μl | 1 | μl |
| H2O, Amgen/Accugene | 36-39 | μl |
| Genomic DNA Template | 2-5 | μl |
| TOTAL VOLUME | 50 | μl |

TABLE 3
PCR reaction conditions used to amplify P2B gene region in
Candida and P2 gene region in Aspergillus spp.
PCR Thermal profile

| Step | Temp | Time | |
|---|---|---|---|
| 1 | 94° C. | 1 min | X 35 |
| 2 | 50° C. | 1 min | |
| 3 | 72° C. | 1 min | |
| 4 | 72° C. | 7 min | |
| 5 | 8° C. | Hold | |

Lid preheating was ON

TABLE 4
TaqMan probes (5′-FAM and 3′-BHQ1 labels)
based on the P2B/P2 gene for C. glabrata
and A. fumigatus.

| Probe Name | Probe Sequence 5′to 3′ |
|---|---|
| P1-Cglab-P2B | CAAGAAGGTTATCGAATCTGTTGGTATTG |
| P1-AspP2 | CCTGCCGCTGCCGGTGCCGCTGCC |
| P2FumP | ACTACAGCTCGAAGATTA |
| P2FlavP | ACGTTGAATGATTGAGAC |
| P2NigP | TTGCGATTACAAGATGGAA |
| P2TerrP | CTTCGGACTACTGCGATGA |
| CAP2BP | ACCGCTTTATTGGAATCCGTTG |
| CKP2BP | ATCCGACAAGTTAGACAAGTTAATC |
| CGP2BP | AGAATCAACGAATTGTTGTCTGC |
| CTP2BP | ATCTTCCAAATTAGACTTATTGTTGA |
| CPP2BP | GAAGAATCAAGATTATCTACCTTGTTG |

TABLE 5
Real-time PCR reagents

| | SAMPLE | |
|---|---|---|
| Preparation of PCR Reaction Mix | x1 | |
| HybProb mix 10x conc. (LightCycler ® FastStartDNA | 2 | μl |
| Master HybProbe Kit) | | |
| MgCl₂ stock solution (Final conc. in reaction is 3 mM) | 1.6 | μl |
| Probe P1-CglabP2B or P1-AspP2 | 2 | μl |
| Primer Forward CglabP2B-F or AspP2-F | 1 | μl |
| Primer Reverse CglabP2B-R or AspP2-R | 1 | μl |
| H₂O PCR-grade | 10.4 | μl |
| Genomic DNA Template | 2 | μl |
| TOTAL VOLUME | 20 | μl |

TABLE 6
Real-time PCR thermocycling conditions:
PCR Thermal profile

| Cycle | Step | Temp | | Time | | |
|---|---|---|---|---|---|---|
| Activation | 1 | 95° | C. | 10 | min | X 50 |
| Amplification | 1 | 95° | C. | 10 | sec | |
| | 2 | 62 or 65° | C. | 20 | sec | |
| | 3 | 70° | C. | 10 | sec | |

| | | | | |
|---|---|---|---|---|
| Cooling | 1 | 40° | C. | Hold |

The PCR was performed with LightCycler ® Roche PCR

### Results

### Primer and Probe Design

The publicly available sequence information for the P2B gene in Candida spp. was aligned with the newly generated sequence information for the P2B gene in Candida spp. and analysed using bioinformatics tools. The sequence information available for the P2 gene in Aspergillus spp. was aligned with the newly generated sequence information for the P2 gene in Aspergillus spp. and analysed using bioinformatics tools. Species-specific probes were designed based on the compiled P2B and P2 sequence information for Candida glabrata and Aspergillus fumigatus respectively (Table 4). FIGS. 3 and 4 show the relative positions of the PCR primers and TaqMan DNA probes for the amplification and detection of C. glabrata and A. fumigatus respectively.

### Real-time PCR

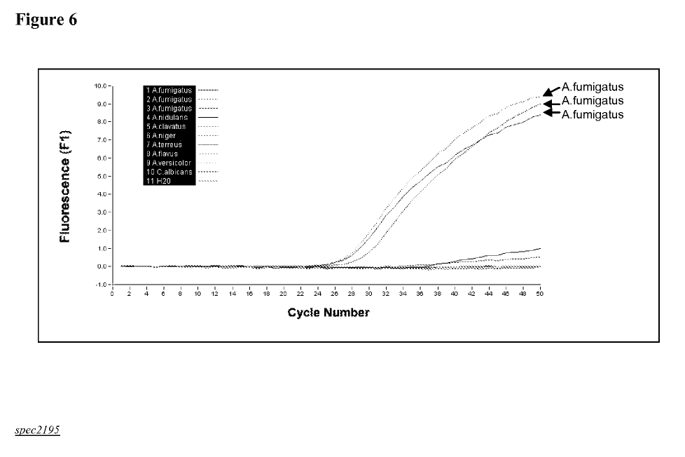

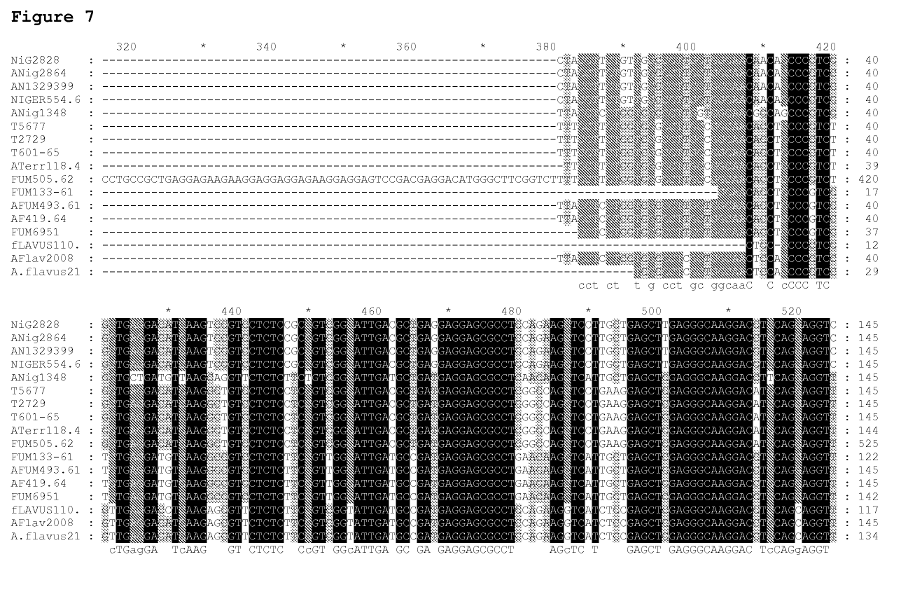

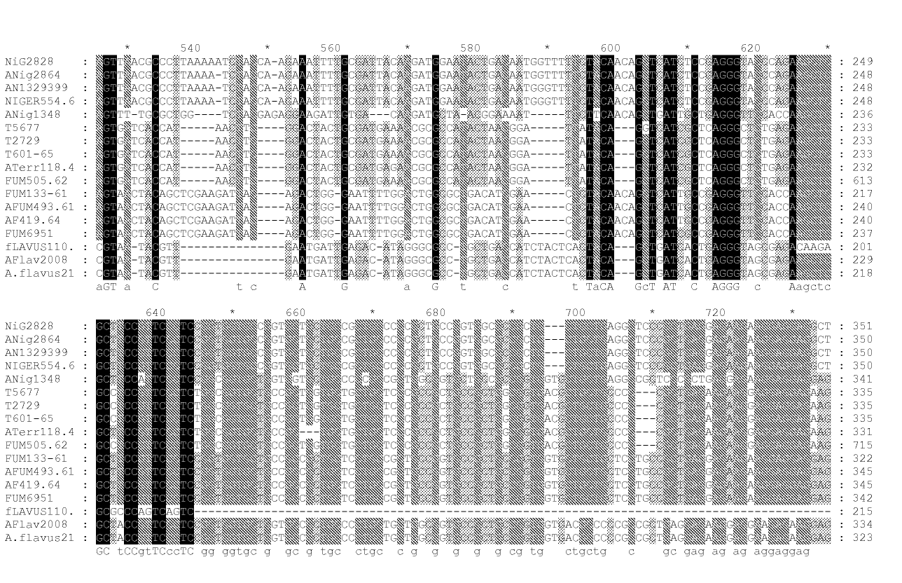

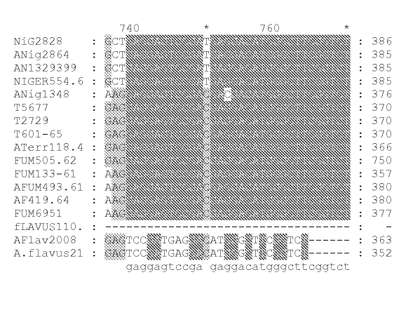

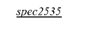

These probes were designed as TaqMan probes and demonstrated for species detection in real-time PCR on the LightCycler using reagents and thermocycling conditions detailed in Tables 5 and 6. For the C. glabrata real-time PCR assay based on the P2B gene, PCR primers CglabP2B-F/CglabP2B-R were combined with TaqMan probe P1-Cglab-P2B. The specificity of the assay for the detection of C. glabrata was confirmed by including DNA from a range of closely related Candida species and A. fumigatus in the C. glabrata real-time PCR assay. The assay detected C. glabrata but did not detect or cross-react with DNA from any other Candida species tested or with A. fumigatus DNA. FIG. 5 shows the results of the C. glabrata real-time PCR assay and the specificity of the assay for C. glabrata. For the A. fumigatus real-time PCR assay based on the P2 gene, PCR primers AspP2-F/AspP2-R were combined with TaqMan probe P1-AspP2. The specificity of the assay for the detection of A. fumigatus was confirmed by including DNA from a range of closely related Aspergillus species and C. albicans in the A. fumigatus real-time PCR assay. The assay detected A. fumigatus but did not detect or cross-react with DNA from C. albicans or any of the four Aspergillus species tested. Two additional Aspergillus spp. did show a small amount of cross reaction in the A. fumigatus real-time PCR assay but this can be eliminated with further assay optimisation. FIG. 6 shows the results of the A. fumigatus real-time PCR assay.

In so far as any sequence disclosed herein differs from its counterpart in the attached sequence listing in PatentIn3.3 software, the sequences within this body of text are to be considered as the correct version.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

### SEQ IDs

Sites of probes, oligonucleotides etc. are shown in bold and underlined. N or x=any nucleotide; w=a/t, m=a/c, r=a/g, k=g/t, s=c/g, y=c/t, h=a/t/c, v=a/g/c, d=a/g/t, b=g/t/c. In some cases, specific degeneracy options are indicated in parenthesis: e.g.: (a/g) is either A or G.

SEQ ID NO 1:
CglabP2B-F
ATTGTTGACCCAAGGTGGTAAC
SEQ ID NO 2:
CglabP2B-R
TCGTCCAAGGACTTACCTTCCAA
SEQ ID NO 3:
CanP2B-F
ATGAAATACTTAGCTGCTTAC
SEQ ID NO 4:
CanP2B-R
TAATCGAATAAACCGAAACCCA
SEQ ID NO 5:
Can P2BflR1
GCGACAAGCAATTTCTCT
SEQ ID NO 6:
Can P2BinR1
ATCATCATCAGATTCTTCTTTG
SEQ ID NO 7:
AspP2-F
ATGAAGCACCTCGCCGC
SEQ ID NO 8:
AspP2-R
AGACCGAAGCCCATGTC
SEQ ID NO 9:
P1-Cglab-P2B
CAAGAAGGTTATCGAATCTGTTGGTATTG
SEQ ID NO 10:
P1-AspP2
CCTGCCGCTGCCGGTGCCGCTGCC
SEQ ID NO 11:
>glab2897\(PF) sequences generated for C.glabrata
CTATTGTTGACCCAAGGTGGTAACGAATCTCCAGCTGCTGCTGACATC
AAGAAGGTTATCGAATCTGTTGGTATTGAAGCTGACGAAGCCAGAATC
AACGAATTGTTGTCTGCTTTGGAAGGTAAGTCCTTGGACGAATTGATC
GCTGAAGGTCAACAAAAGTTCGCCTCTGTTCCAGTTGGTGGTGCTGCT
GCTGGTGGTGCTTCCGCTGCTGCTGGTGGTGCCGCTGCCGGTGAAGCC
GCTGAAGAAAAGGAAGAAGAAGCTGCTGAAGAATCCGATGACGACATG
GGTTTCGGTTTATTCGATTA
SEQ ID NO 12:
>glab8018\(PF) sequences generated for C.glabrata
TTGTTGACCCAAGGTGGTAACGAATCTCCAGCTGCTGCTGACATCAAG
AAGGTTATCGAATCTGTTGGTATTGAAGCTGACGAAGCCAGAATCAAC
GAATTGTTGTCTGCTTTGGAAGGTAAGTCCTTGGACGAATTGATCGCT
GAAGGTCAACAAAAGTTCGCCTCTGTTCCAGTTGGTGGTGCTGCTGCT
GGTGGTGCTTCCGCTGCTGCTGGTGGTGCCGCTGCCGGTGAAGCCGCT
GAAGAAAAGGAAGAAGAAGCTGCTGAAGAATCCGATGACGACATGGGT
TTCGGTTTATTCGATTA
SEQ ID NO 13:
>glab4692\(PF) sequences generated for C.glabrata
CTATTGTTGACCCAAGGTGGTAACGAATCTCCAGCTGCTGCTGACATC
AAGAAGGTTATCGAATCTGTTGGTATTGAAGCTGACGAAGCCAGAATC
AACGAATTGTTGTCTGCTTTGGAAGGTAAGTCCTTGGACGAATTGATC
GCTGAAGGTCAACAAAAGTTCGCCTCTGTTCCAGTTGGTGGTGCTGCT
GCTGGTGGTGCTTCCGCTGCTGCTGGTGGTGCCGCTGCCGGTGAAGCC
GCTGAAGAAAAGGAAGAAGAAGCTGCTGAAGAATCCGATGACGACATG
GGTTTCGGTTTATTCGATTA
SEQ ID NO 14:
>glabl38\(PF) sequences generated for C.glabrata
CTATTGTTGACCCAAGGTGGTAACGAATCTCCAGCTGCTGCTGACATC
AAGAAGGTTATCGAATCTGTTGGTATTGAAGCTGACGAAGCTAGAATC
AACGAATTGTTGTCTGCTTTGGAAGGTAAGTCCTTGGACGAATTGATC
GCTGAAGGTCAACAAAAGTTCGCCTCTGTTCCAGTTGGTGGTGCTGCT
GCTGGTGGTGCTTCCGCTGCTGCTGGTGGTGCCGCTGCCGGTGAAGCT
GCTGAAGAAAAGGAAGAAGAAGCTGCTGAAGAATCCGATGACGACATG
GGTTTCGGTTTATTCGATTA
SEQ ID NO 15:
>krusei3165\(P2BF) sequence generated for C. krusei
TTACTCTTAGTCAATGCTGGTAAAACCGCACCATCTGCTGCAGATGTT
ACCTCTGTCTTATCATCTGTCGGTATCGAAGTTGAATCCGACAAGTTA
GACAAGTTAATCTCCGAATTAGAAGGTAAGTCCGTCGAAGAGTTGATT
GCTGAAGGTACTGAAAAGATGGCTTCTGCTCCAGGTGCAGCAGCTGCT
CCAGCTTCTGGTGCAGGTGCTTCCACCGAATCTGCTGCAGCAGAAGAA
GTTGAAGAAGAAAAGGAAGAATCCGATGATGACATGGGTTTCGGTTTA
TTCGATTA
SEQ ID NO 16:
>krusei9560\(P2BF) sequence generated for C. krusei
CATCTGCTGCAGATGTTACCTCTGTCTTATCATCTGTCGGTATCGAAG
TTGAATCCGACAAGTTAGACAAGTTAATCTCCGAATTAGAAGGTAAGT
CCGTCGAAGAGTTGATTGCTGAAGGTACTGAAAAGATGGCTTCTGCTC
CAGGTGCAGCAGCTGCTCCAGCTTCTGGTGCAGGTGCTTCCACCGAAT
CTGCTGCAGCAGAAGAAGTTGAAGAAGAAAAGGAAGAATCCGATGATG
ACATGGGTTTCGGTTTATTCGATTA
SEQ ID NO 17:
>krusei60554P2BF) sequence generated for C. krusei
CATCTGCTGCAGATGTTACCTCTGTCTTATCATCTGTCGGTATCGAAG
TTGAATCCGACAAGTTAGACAAGTTAATCTCCGAATTAGAAGGTAAGT
CCGTCGAAGAGTTGATTGCTGAAGGTACTGAAAAGATGGCTTCTGCTC
CAGGTGCAGCAGCTGCTCCAGCTTCTGGTGCAGGTGCTTCCACCGAAT
CTGCTGCAGCAGAAGAAGTTGAAGAAGAAAAGGAAGAATCCGATGATG
ACATGGGTTTCGGTTTATTCGATTA
SEQ ID NO 18:
>krusei573\(P2B) sequence generated for C. krusei
TTACTCTTAGTACATGCTGGTAAAACCGCACCATCTGCTGCAGATGTT
ACCTCTGTCTTATCATCTGTCGGTATCGAAGTTGAATCCGACAAGTTA
GACAAGTTAATCTCCGAATTAGAAGGTAAGTCCGTCGAAGAGTTGATT
GCTGAAGGTACTGAAAAGATGGCTTCTGCTCCAGGTGCAGCAGCTGCT
CCAGCTTCTGGTGCAGGTGCTTCCACCGAATCTGCTGCAGCAGAAGAA
GTTGAAGAAGAAAAGGAAGAATCCGATGATGACATGGGTTTCGGTTTA
TTCGATTA
SEQ ID NO 19:
>CA562P2B sequence generated for C.albicans
TTATTGTTAGTTCAAGGTGGTAACACCTCTCCATCAGCTTCTGATATC
ACCGCTTTATTGGAATCCGTTGGTGTTGAAGCCGAAGAATCCAGATTA
CAAGCTTTATTGAAAGATTTGGAAGGTAAAGACTTGCAAGAATTGATT
GCTGAAGGTAACACCAAATTAGCTTCTGTCCCATCCGGTGGTGCTGCT
GCTGGTGGTGCTTCTGCCTCTGCTGGTGCCGCTGCTGGTGGTGCTGCT
GAAGCTGAAGAAGAAAAAGAAGAAGAAGCCAAAGAAGAATCTGATGAT
GATATGGGTTTCGGTTTATTCGATTAGAGAAATTGCTTGTCGC
SEQ ID NO 20:
>A765\(P2BF) P2B sequence generated for C.albicans
TTATTGTTAGTTCAAGGTGGTAACACCTCTCCATCAGCTTCTGATATC
ACCGCTTTATTGGAATCCGTTGGTGTTGAAGCCGAAGAATCCAGATTA
CAAGCTTTATTGAAAGATTTGGAAGGTAAAGACTTGCAAGAATTGATT
GCTGAAGGTAACACCAAATTAGCTTCTGTCCCATCCGGTGGTGCTGCT
GCTGGTGGTGCTTCTGCCTCTGCTGGTGCCGCTGCTGGTGGTGCTGCT
GAAGCTGAAGAAGAAAAAGAAGAAGAAGCCAAAGAAGAATCTGATGAT
GATATGGGTTTCGGTTTATTCGATTAGAGAAATTGCTTGTCGC
SEQ ID NO 21:
>A3156\(P2BF) P2B sequence generated for C.albicans
TTATTGTTAGTTCAAGGTGGTAACACCTCTCCATCAGCTTCTGATATC
ACCGCTTTATTGGAATCCGTTGGTGTTGAAGCCGAAGAATCCAGATTA
CAAGCTTTATTGAAAGATTTGGAAGGTAAAGACTTGCAAGAATTGATT
GCTGAAGGTAACACCAAATTAGCTTCTGTCCCATCCGGTGGTGCTGCT
GCTGGTGGTGCTTCTGCCTCTGCTGGTGCCGCTGCTGGTGGTGCTGCT
GAAGCTGAAGAAGAAAAAGAAGAAGAAGCCAAAGAAGAATCTGATGAT
GATATGGGTTTCGGTTTATTCGATTAGAGAAATTGCTTGTCGC
SEQ ID NO 22:
>A2700\(P2BF) P2B sequence generated for C.albicans
TTATTGTTAGTTCAAGGTGGTAACACCTCTCCATCAGCTTCTGATATC
ACCGCTTTATTGGAATCCGTTGGTGTTGAAGCYGAAGAATCCAGATTA
CAAGCTTTATTGAAAGATTTGGAAGGTAAAGACTTGCAAGAATTGATT
GCTGAAGGTAACACCAAATTAGCTTCTGTCCCATCCGGTGGTGCTGCT
GCTGGTGGTGCTTCTGCCTCTRCTGGTGCCGCTGCTGGYGGTGCTGCY
GAAGCTGAAGAAGAAAAAGAAGAAGAAGCCAAAGAAGAATCTGATGAT
GATATGGGTTTCGGTTTATTCGATTAGAGAAATTGCTTGTCGC
SEQ ID NO 23:
>D3949\(P2BF) P2B sequence generated for
C.dubliniensis
TTGTTGTTAGTTCAAGGTGGTAACGCCACTCCATCAGCTTCTGATATC
AGCGCTGTCTTGGAAACTGTTGGTGTTGAAGCCGAAGAATCCAGATTA
CAAGCTTTATTGAAAGATTTGGAAGGTAAAGATTTGCAAGAATTGATT
GCTGAAGGTAACACCAAATTAGCTTCTGTCCCAACCGGTGGTGCTGCT
GCTGGTGGTGCTTCCGGTTCTGCTGGTGCCGCTTCTGGTGCCGCTGCT
GAAGCTGAAGAAGAAAAAGAAGAAGAAGCTAAAGAAGAATCTGATGAT
GATATGGGTTTCGGTTTATTCGATTAGAGAAATTGCTTGTCGC
SEQ ID NO 24:
>D7987\(P2BF) sequence generated for
C.dubliniensis
TTGTTGTTAGTTCAAGGTGGTAACGCCACTCCATCAGCTTCTGATATC
AGCGCTGTCTTGGAAACTGTTGGTGTTGAAGCCGAAGAATCCAGATTA
CAAGCTTTATTGAAAGATTTGGAAGGTAAAGATTTGCAAGAATTGATT
GCTGAAGGTAACACCAAATTAGCTTCTGTCCCAACCGGTGGTGCTGCT
GCTGGTGGTGCTTCCGGTTCTGCTGGTGCCGCTTCTGGTGCCGCTGCT
GAAGCTGAAGAAGAAAAAGAAGAAGAAGCCAAAGAAGAATCTGATGAT
GATA
SEQ ID NO 25:
>CP604\(CanP2B-FOR>><<REV) P2B sequences
generated for C.parapsilosis
ATGAAATACTTAGCTGCTTACTTATTATTGGTCCAAGGTGGTAACGCC
TCCCCATCTGCTTCAGACATCTCCTCATTGTTGGAATCCGTTGGTGTT
GAAGTTGAAGAATCAAGATTATCTACCTTGTTGAAAGACTTGGAAGGT
AAAGACATCAACGAGTTGATTGCTGAAGGTAACACCAAATTGGCCTCA
GTTCCATCTGGTGGTGCTGCTGTTGCTTCCGGTTCTGGTGCTTCTGGT
GCCGCTGCTGGTGGTGCTGCTGAAGAAGCTAAGGAAGAAGCCAAGGAA
GAAGAAAAGGAAGAATCTGATGATGACATGGGTTTCGGTTTATTCGAT
TA
SEQ ID NO 26:
>P2194\(P2BF) sequences generated for
C.parapsilosis
TTATTATTGGTCCAAGGTGGTAACGCCTCCCCATCTGCTTCAGACATC
TCCTCATTGTTGGAATCCGTTGGTGTTGAAGTTGAAGAATCAAGATTA
TCTACCTTGTTGAAAGACTTGGAAGGTAAAGACATCAACGAGTTGATT
GCTGAAGGTAACACCAAATTGGCCTCAGTTCCATCTGGTGGTGCTGCT
GTTGCTTCCGGTTCTGGTGCTTCTGGTGCCGCTGCTGGTGGTGCTGCT
GAAGAAGCTAAGGAAGAAGCCAAGGAAGAAGAAAAGGAAGAATCTGAT
GATGACATGGGTTTCGGTTTATTCGATTA
SEQ ID NO 27:
>P96141\(P2BF) sequences generated for
C.parapsilosis
GGTGGTAACGCCTCCCCATCCGCTTCAGACATCTCATCCTTGTTGGAA
TCCGTTGGTGTTGAAGTTGAAGAATCAAGATTGTCCCTCTTGTTGAAA
GACTTGGAAGGTAAAGACATCAACGAATTGATTGCTGAAGGTAACACC
AAGTTGGCTTCAGTTCCAACTGGTGGTGCTGCTGTTGCTTCTGGTTCT
GGTGCTTCAGGTGCCGCTGCTGGTGGTGCTGCTGAAGAAGCCAAAGAA
GAATCTGATGATGAT
SEQ ID NO 28:
>CT94\(P2BF) sequences generated for C.tropicalis
GGTGGTAACGCTTCCCCATCTGCTTCTGACATCTCCGCTTTATTGGAA
CAAGTTGGTGCTGAAGTTGAATCTTCCAAATTAGACTTATTGTTGAAA
GAATTGGAAGGTAAAGACTTGCAAGAATTGATTGCCGAAGGTAACACT
AAATTCGCCTCTGTCCCATCCGGTGGTGCTGCTGCTGCTTCYTCTGGT
TCCGCTGCCGCTGCTGGTGGTGCCGCTGCCGAAGCTGAAGAAGAAAAA
GAAGAAGAAGCCAAAGAAGAATCTGATGATGAT
SEQ ID NO 29:
>CT2311\(P2BF) sequences generated for
C.tropicalis
GGTGGTAACGCTTCCCCATCTGCTTCTGACATCTCCGCTTTATTGGAA
CAAGTTGGTGCTGAAGTTGAATCTTCCAAATTAGACTTATTGTTGAAA
GAATTGGAAGGTAAAGACTTGCAAGAATTGATTGCCGAAGGTAACACT
AAATTCGCCTCTGTCCCATCCGGTGGTGCTGCTGCTGCTTCCTCTGGT
TCCGCTGCCGCTGCTGGTGGTGCCGCTGCCGAAGCTGAAGAAGAAAAA
GAAGAAGAAGCCAAAGAAGAATCTGATGATGAT
SEQ ID NO 30:
>T-8157\(PF) sequences generated for C.tropicalis
TTATTATTAGTCCAAGGTGGTAACRCTTCCCCATCTGCTTCTGACATC
TCCGCTTTATTGGAACAAGTTGGTGCTGAAGTTGAATCTTCCAAATTA
GACTTATTGTTGAAAGAATTGGAAGGTAAAGACTTGCAAGAATTGATT
GCCGAAGGTAACACTAAATTCGCCTCTGTCCCATCCGGTGGTGCTGCY
GCTGCTTCCTCTGGTTCCGCTGCCGCTGCTGGTGGTGCCGCTGCCGAA
GCTGAAGAAGAAAAAGAAGAAGAAGCCAAAGAAGAATCTGATGATGAT
SEQ ID NO 31:
>T-2424\(PF) sequences generated for C.tropicalis
TTATTATTAGTCCAAGGTGGTAACGCTTCCCCATCTGCTTCTGACATC
TCCGCTTTATTGGAACAAGTTGGTGCTGAAGTTGAATCTTCCAAATTA
GACTTATTGTTGAAAGAATTGGAAGGTAAAGACTTGCAAGAATTGATT
GCCGAAGGTAACACTAAATTCGCCTCTGTCCCATCCGGTGGTGCTGCT
GCTGCTTCCTCTGGTTCCGCTGCCGCTGCTGGTGGTGCCGCTGCCGAA
GCTGAAGAAGAAAAAGAAGAAGAAGCCAAAGAAGAATCTGATGATGAT
SEQ ID NO 32:
>T-3895\(PF) sequences generated for C.tropicalis
TTATTATTAGTCCAAGGTGGTAACGCTTCCCCATCTGCTTCTGACATC
TCCGCTTTATTGGAACAAGTTGGTGCTGAAGTTGAATCTTCCAAATTA
GACTTATTGTTGAAAGAATTGGAAGGTAAAGACTTGCAAGAATTGATT
GCCGAAGGTAACACTAAATTCGCCTCTGTCCCATCCGGTGGTGCTGCT
GCTGCTTCTTCTGGTTCCGCTGCCGCTGCTGGTGGTGCCGCTGCCGAA
GCTGAAGAAGAAAAAGAAGAAGAAGCCAAAGAAGAATCTGATGATGAT
SEQ ID NO 33:
>guill2672\(P2BF) sequences generated for
C.guilliermondii
TTRTTGTTGGTKRACGCCGGTAACACCTCCCCATCTGCTGCTGACATC
AAGGCTGTCTTGGAGTCGGTTTCCATAGAAGTTGACGACGAGAAGGTG
TCCAAGTTGTTGAGCGAAGTTGAGGGAAAGAATGCTGAAGAATTGATC
GCTGAAGGTAACGAAAAATTGTCTTCTGTTCCAACTGGTGGACCAGCT
GCTGCTTCCTCTGGATCTGCTGCCGCTGCCGATGCTCCTGCTGCCGAA
GAGGCCGCTGAGGAGGCCGCTGAGGAGTCTGACGACGACATGGGTTTC
GGTTTATTCGATTA
SEQ ID NO 34:
>guill6021\(P2BF) sequences generated for
C.guilliermondii
TTRTTGTTGGTKRACGCCGGTAACACCTCCCCATCTGCTGCTGACATC
AAGGCTGTCTTGGAGTCGGTTTCCATAGAAGTTGACGACGAGAAGGTG
TCCAAGTTGTTGAGCGAAGTTGAGGGAAAGAATGCTGAAGAATTGATC
GCTGAAGGTAACGAAAAATTGTCTTCTGTTCCAACTGGTGGACCAGCT
GCTGCTTCCTCTGGATCTGCTGCCGCTGCCGATGCTCCTGCTGCCGAA
GAGGCCGCTGAGGAGGCCGCTGAGGAGTCTGACGACGACATGGGTTTC
GGTTTATTCGATTA
SEQ ID NO 35:
>lus7270\(P2BF) sequences generated for
C.lusitanie
TTATTGTTGGTCAACGCTGGTAACACCGCCCCATCTGCTGCTGACGTC
AAGAAGGTCTTGGAATCCGTCTCTATTGAGGTTGAGGACGACAAGGTT
GAGAAGTTGTTGGCTGAAGTTGAAGGCAAGAACGTCGAAGAGTTGATT
GCCGAGGGTAACGAGAAGTTGTCTTCTGTTCCATCTGGTGCTCCAGCT
GCTGCTGGTGCCGCTGCTGCTTCTGGTTCTACTGAGGCTGCTGCTGAA
GAGCCACAAGAAGAAGAGAAGGAGGAGTCTGACGACGACATGGGTTTC
GGTTTATTCGATTA
SEQ ID NO 36:
>AF419.64\(AspP2-F) P2 sequences generated for
A.fumigatus
TTACCTCCTCCTCGCCCTTGCTGGCAACACCTCCCCGTCCTCTGAGGA
TGTCAAGGCCGTCCTCTCTTCCGTTGGCATTGATGCCGATGAGGAGCG
CCTGAACAAGCTCATTGCTGAGCTCGAGGGCAAGGACCTCCAGGAGGT
TAGTAACTACAGCTCGAAGATTACAGACTGGGAATTTTGGACTGGCGC
TGACATCGAACTCTACAACAGCTCATTGCCGAGGGTTCCACCAAGCTC
GCTTCCGTTCCCTCCGGTGGTGCTGCCGCCGCTGCTCCTGCCGCTGCC
GGTGCCGCTGCCGGTGGTGCTGCTGCTCCTGCCGCTGAGGAGAAGAAG
GAGGAGGAGAAGGAGGAGTCCGACGAGGACATGGGCTTCGGTCT
SEQ ID NO 37:
>F-6951\(APF) P2 sequences generated for
A.fumigatus
CCTCCTCCTCGCCCTTGCTGGCAACACCTCCCCGTCCTCTGAGGATGT
CAAGGCCGTCCTCTCTTCCGTTGGCATTGATGCCGATGAGGAGCGCCT
GAACAAGCTCATTGCTGAGCTCGAGGGCAAGGACCTCCAGGAGGTTAG
TAACTACAGCTCGAAGATTACAGACTGGGAATTTTGGACTGGCGCTGA
CATCGAACTCTACAACAGCTCATCGCCGAGGGTTCCACCAAGCTCGCT
TCCGTTCCCTCCGGTGGTGCTGCCGCCGCTGCTCCTGCCGCTGCCGGT
GCCGCTGCCGGTGGTGCTGCTGCTCCTGCCGCTGAGGAGAAGAAGGAG
GAGGAGAAGGAGGAGTCCGACGAGGACATGGGCTTCGGTCT
SEQ ID NO 38:
>F-133-61\(APF) P2 sequences generated for
A.fumigatus
GCAACACCTCCCCGTCCTCTGAGGATGTCAAGGCCGTCCTCTCTTCCG
TTGGCATTGATGCCGATGAGGAGCGCCTGAACAAGCTCATTGCTGAGC
TCGAGGGCAAGGACCTCCAGGAGGTTAGTAACTACAGCTCGAAGATTA
CAGACTGGGAATTTTGGACTGGCGCTGACATCGAACTCTACAACAGCT
CATTGCCGAGGGTTCCACCAAGCTCGCTTCCGTTCCCTCCGGTGGTGC
TGCCGCCGCTGCTCCTGCCGCTGCCGGTGCCGCTGCCGGTGGTGCTGC
TGCTCCTGCCGCTGAGGAGAAGAAGGAGGAGGAGAAGGAGGAGTCCGA
CGAGGACATGGGCTTCGGTCT
SEQ ID NO 39:
>AF493.61\(Asp2F) P2 sequences generated for
A.fumigatus
TTACCTCCTCCTCGCCCTTGCTGGCAACACCTCCCCGTCCTCTGAGGA
TGTCAAGGCCGTCCTCTCTTCCGTTGGCATTGATGCCGATGAGGAGCG
CCTGAACAAGCTCATTGCTGAGCTCGAGGGCAAGGACCTCCAGGAGGT
TAGTAACTACAGCTCGAAGATTACAGACTGGGAATTTTGGACTGGCGC
TGACATCGAACTCTACAACAGCTCATTGCCGAGGGTTCCACCAAGCTC
GCTTCCGTTCCCTCCGGTGGTGCTGCCGCCGCTGCTCCTGCCGCTGCC
GGTGCCGCTGCCGGTGGTGCTGCTGCTCCTGCCGCTGAGGAGAAGAAG
GAGGAGGAGAAGGAGGAGTCCGACGAGGACATGGGCTTCGGTCT
SEQ ID NO 40:
>AF1085-P2\(AspP2-F) P2 sequences generated for
N.fischeri
TTACCTCCTCCTCGCCCTTGCTGGCAACACCTCCCCCTCCGCTGAGGA
TGTCAAGGCCGTCCTCTCTTCCGTCGGCATTGACGCCGATGAGGAGCG
CCTGAACAAGCTCATTGCTGAGCTCGAGGGCAAGGACCTCCAGGAGGT
TAGTACACACGGCTTGAATATTACCGACTGAGAATTTTGGACCGGCGC
TGACATCGATTTCTACAACAGCTGATCGCTGAGGGTTCCGCCAAGCTC
GCTTCCGTTCCCTCCGGTGGTGCCGGTGGTGCCGCTGCTCCTGCCGCT
GGCGGTGCCGCTGCCGGTGGTGCTGCTGCCGCTCCCGCCGAAGAGAAG
GAGGAGGAGAAGGAGGAGTCCGACGAGGACATGGGCTTCGGTCT
SEQ ID NO 41:
>Nid100-2\(P2F) P2B sequence for A. nidulans
CTACCTCCTCCTCGCCCTTGCTGGCAACGAGTCTCCCTCCGCCTCCGA
CATCAAGGAGGTTCTCTCTTCCGTCGGTGTTGACGCCGACGACGAGCG
CCTCGAGAAGCTCATTGCTGAGCTCCAGGGCAAGGACATCAACGAGGT
TCGTTATTGCATATAGAGTTGGAAGACGCGGACTGCGGGCTAACGATA
ATCTTTAAACAGCTGATCGCTGAGGGTACCACCAAGCTTGCCTCCGTT
CCCTCCGGCGGTGCTGGTGGTGCTGCCCCTGCTGCCGCTGCCGGTGGT
GCTGCTGCTGCCGAGGCCCCCGCTGCTGAGAAGGAGGAGGAGAAGGAG
GAGTCCGATGAGGACATGGGCTTCGGTCT
SEQ ID NO 42:
>Nid7063\(P2F) P2B sequence for A. nidulans
CCTTGCTGGCAACGAGTCTCCCTCCGCCTCCGACATCAAGGAGGTTCT
CTCTTCCGTCGGTGTTGACGCCGACGACGAGCGCCTCGAGAAGCTCAT
TGCTGAGCTCCAGGGCAAGGACATCAACGAGGTTCGTTATTGCATAGG
GTTGGAAGACGCGGACAGCGGGCTAACGATAATCTTCTGAACAGCTGA
TCGCTGAGGGTACCACCAAGCTTGCCTCCGTTCCCACCGGCGGTGCTG
GTGCTGCTGCCCCTGCTGCCGCTGCCGGTGGTGCTGCCGCTGCCGAGG
CTCCCGCTGCTGAGAAGGAGGAGGAGAAGGAGGAGTCCGATGAGGACA
TGGGCTTCGGTCT
SEQ ID NO 43:
>AC5138\(AspP2-F) P2 sequences generated for
A.clavatus
TTACCTCCTCCTCGCCCTTGGTGGCAACGCCAGCCCCTCCGCTGCTGA
TGTTAAGGAGGTTCTCTCTTCTGTCGGCATTGATGCTGATGAGGAGCG
CCTCAACAAGCTCATTGCTGAGCTCGAGGGCAAGGACCTTCAGGAGGT
TAGTTTTGCGCTGGTCTACGAGAGGAAGATTGTGACAAGATGCTAACG
GAAAATTTCTTCAACAGCTGATTGCTGAGGGTTCCACCAAGCTCGCTT
CCATTCCCTCCGGCGGTGCTGGTGGTGCTGCCCCCGCCGCTGGCGGTG
CTGCCGCCGGTGGTGCTGCTGAGGCCGCTCCCGCTGAGGAGAAGGAGG
AGGAGAAGGAGGAGTCCGACGACGACATGGGCTTCGGTCT
SEQ ID NO 44:
>AN1329399-P2\(AspP2-F) P2 sequences generated
for A.niger
CTACCTTCTGTTGGCCCTTGCTGGCAACAACACCCCCTCCGCTGAGGA
CATCAAGTCCGTCCTCTCCGCCGTCGGCATTGACGCTGAGGAGGAGCG
CCTCCAGAAGCTCCTTGCTGAGCTTGAGGGCAAGGACCTCCAGGAGGT
CAGTTAACGCCCTTAAAATCTACCAAGAAATTTTGCGATTACAAGATG
GAATACTGACAATGGGTTTTCTACAACAGCTCATCTCCGAGGGTACCC
AGAAGCTCGCTTCCGTTCCCTCCGGTGGTGCCGGTGCTGCTGCCGCTG
CCCCCGCTGCCGGTGGCGCCGCTGCTGCTGAGGCTCCCGCTGAGGAGA
AGAAGGAGGAGGCTGCTGAGGAGTCCGATGAGGACATGGGCTTCGGTC
T
SEQ ID NO 45:
>AN2864\(Asp2F) sequences generated for A.niger
CTACCTTCTGTTGGCCCTTGCTGGCAACAACACCCCCTCCGCTGAGGA
CATCAAGTCCGTCCTCTCCGCCGTCGGCATTGACGCTGAGGAGGAGCG
CCTCCAGAAGCTCCTTGCTGAGCTTGAGGGCAAGGACCTCCAGGAGGT
CAGTTAACGCCCTTAAAATCTACCAAGAAATTTTGCGATTACAAGATG
GAATACTGACAATGGTTTTTCTACAACAGCTCATCTCCGAGGGTACCC
AGAAGCTCGCTTCCGTTCCCTCCGGTGGTGCCGGTGCTGCTGCCGCTG
CCCCCGCTGCCGGTGGCGCCGCTGCTGCTGAGGCTCCCGCTGAGGAGA
AGAAGGAGGAGGCTGCTGAGGAGTCCGATGAGGACATGGGCTTCGGTC
T
SEQ ID NO 46:
>AT118.46\(AspP2 -F) P2 sequences generated for
A.terreus
TTCCTTCTCCTCGGCCTTGCCGGCAACACCTCCCCCTCTGCTGAGGAC
ATCAAGGCTGTCCTCTCCTCCGTCGGCATTGACGCTGATGAGGAGCGC
CTCGGCCAGCTCCTGAAGGAGCTCGAGGGCAAGGACATCCAGGAGGTT
AGTGATCACCATAACTTCGGACTACTGCGATGAGAACGCGCCATACTA
ACGGATTATACAGCTCATCGCTCAGGGCTCTGAGAAGCTCGCTTCCGT
TCCCTCTGGCGGTGCTGCCGCTGCTGCTGCTCCCGCCGCTGCCGCTGG
CGGTGACGCTGCTGCCCCCGCTGAGAAGAAGGAGGAGGAGAAGGAGGA
GGAGTCCGACGAGGACATGGGCTTCGGTCT
SEQ ID NO 47:
>AF2008\(AspP2-F) P2 sequences generated for
A.flavus
TTACCTCCTCCTCGCCCTCGCTGGCAACTCCACCCCCTCCGTTGAGGA
CATCAAGAGCGTTCTCTCTTCCGTCGGTATTGATGCCGATGAGGAGCG
CCTCCAGAAGGTCATCTCCGAGCTCGAGGGCAAGGACCTCCAGCAGGT
TCGTAATACGTTGAATGATTGAGACATAGGGCGCCTGCTGACCATCTA
CTCACTACAGCTGATCACTGAGGGTAGCGAGAAGCTCGCTACCGTTCC
CTCCGGTGGTGCTGGTGCCGCTGCCCCTGCTGCTGGCGGTGCCGCTGC
CGGTGGTGACGCCCCCGCCGCTGAGGAGAAGGAGGAAGAGAAGGAGGA
GTCCGATGAGGACATGGGCTTCGGTCT
SEQ ID NO 48:
>AV1323\(AspP2-F) P2 sequences generated for
A.versicolor
TCGCCCTTGCTGGCAACGAGAGCCCCTCTGCTTCCGACATTAAGGAGG
TTCTGTCCTCCGTCGGTGTTGACGCTGACAACGAGCGCCTCGAGAAGC
TCATCGCTGAGCTCCAGGGCAAGGACATCAACGAGGTTCGTTTTGACA
GATGCATTTGAAATACTTGGCCAGCAGACTAATGAAACCTCTTCTGCA
GTTGATCGCTGAGGGTACCACCAAGCTCGCTTCCGTTCCCTCTGGCGG
TGGTGGTGGTGCTGCCGCCCCCGCTGCTGGTGGCGCTGCCGCCGCTGA
GGCCCCTGCTGCTGAGAAGGAGGAGGAGAAGGAGGAGTCCGACGAGGA
ATGGGCTTCGGTCT
SEQ ID NO 49:
>AV2916\(Asp2F) sequences generated for
A.versicolor
CCCTTGCTGGCAACGAGAGCCCCTCTGCTTCCGACATTAAGGAGGTTC
TGTCCTCCGTCGGTGTTGACGCTGACGACGAGCGCCTCGAGAAGCTCA
TCGCTGAGCTCCAGGGCAAGGACATCAACGAGGTTCGTTTTGACAGAT
GCGTTTGAAATACTTGGCCAGCAGACTAATGAAACCTCTTCTGCAGTT
GATCGCTGAGGGTACCACCAAGCTCGCTTCCGTTCCCTCTGGCGGTGG
TGGTGGTGCTGCCGCCCCCGCTGCTGGTGGCGCTGCCGCCGCTGAGGC
CCCTGCTGCTGAGAAGGAGGAGGAGAAGGAGGAGTCCGACGAGGACAT
GGGCTTCGGTCT
SEQ ID NO 50:
>Published P2B sequences for C albicans
gi|11229041|gb|AF317662.1|AF317662
Candida albicans 60S acidic ribosomal protein
type P2-B (p2B) gene, complete cds
GGAAAAAATGAAATACTTAGCTGCTTACTTATTGTTAGTTCAAGGTGG
TAACACCTCTCCATCAGCTTCTGATATCACCGCTTTATTGGAATCCGT
TGGTGTTGAAGCCGAAGAATCCAGATTACAAGCTTTATTGAAAGATTT
GGAAGGTAAAGACTTGCAAGAATTGATTGCTGAAGGTAACACCAAATT
AGCTTCTGTCCCATCCGGTGGTGCTGCTGCTGGTGGTGCTTCTGCCTC
TACTGGTGCCGCTGCTGGTGGTGCTGCCGAAGCTGAAGAAGAAAAAGA
AGAAGAAGCCAAAGAAGAATCTGATGATGATATGGGTTTCGGTTTATT
CGATTAGAGAAATTGCTTGTCGCCTTTGCTGGTTTGAGAGAAGTATAT
TTCCATTATTTTGCATTATATATATATATATGTATTATAACTAATCTA
ATAAAAAAAATATGAAAACAAAAATGGCTTCTATATGGCACTGTTTGC
A
SEQ ID NO 51:
>Published P2B sequences for C.glabrata
gi|50284952|ref|XM_444905.1| Candida
glabrata CBS138 hypothetical protein
(CAGLOA03168g) partial mRNA
ATGAAGTACTTGGCCGCTTACCTATTGTTGACCCAAGGTGGTAACGAA
TCTCCAGCTGCTGCTGACATCAAGAAGGTTATCGAATCTGTTGGTATT
GAAGCTGACGAAGCTAGAATCAACGAATTGTTGTCTGCTTTGGAAGGT
AAGTCCTTGGACGAATTGATCGCTGAAGGTCAACAAAAGTTCGCCTCT
GTTCCAGTTGGTGGTGCTGCTGCTGGTGGTGCTTCCGCTGCTGCTGGT
GGTGCCGCTGCCGGTGAAGCTGCTGAAGAAAAGGAAGAAGAAGCTGCT
GAAGAATCCGATGACGACATGGGTTTCGGTTTGTTCGACTAA
SEQ ID NO 52:
>gi|68465556|ref|XM_718047.1| Candida albicans
SC5314 cytosolic ribosomal acidic protein P2B
(CaO19_5928) partial mRNA
ATGAAATACTTAGCTGCTTACTTATTGTTAGTTCAAGGTGGTAACACC
TCTCCATCAGCTTCTGATATCACCGCTTTATTGGAATCCGTTGGTGTT
GAAGCCGAAGAATCCAGATTACAAGCTTTATTGAAAGATTTGGAAGGT
AAAGACTTGCAAGAATTGATTGCTGAAGGTAACACCAAATTAGCTTCT
GTCCCATCCGGTGGTGCTGCTGCTGGTGGTGCTTCTGCCTCTGCTGGT
GCCGCTGCTGGTGGTGCTGCTGAAGCTGAAGAAGAAAAAGAAGAAGAA
GCCAAAGAAGAATCTGATGATGATATGGGTTTCGGTTTATTCGATTAG
SEQ ID NO 53:
>gi|68465849|ref|XM_717900.1| Candida albicans
SC5314 cytosolic ribosomal acidic protein P2B
(CaO19_13349) partial mRNA
ATGAAATACTTAGCTGCTTACTTATTGTTAGTTCAAGGTGGTAACACC
TCTCCATCAGCTTCTGATATCACCGCTTTATTGGAATCCGTTGGTGTT
GAAGCCGAAGAATCCAGATTACAAGCTTTATTGAAAGATTTGGAAGGT
AAAGACTTGCAAGAATTGATTGCTGAAGGTAACACCAAATTAGCTTCT
GTCCCATCCGGTGGTGCTGCTGCTGGTGGTGCTTCTGCCTCTGCTGGT
GCCGCTGCTGGTGGTGCTGCTGAAGCTGAAGAAGAAAAAGAAGAAGAA
GCCAAAGAAGAATCTGATGATGATATGGGTTTCGGTTTATTCGATTAG
SEQ ID NO 54:
>Published P2 sequence for A.fumigatus
gi|71001323|ref|XM_750250.1| Aspergillus fumigatus
Af293 60S acidic ribosomal protein P2/allergen
Asp F 8 (AFUA_2G10100) mRNA, complete cds
ATGAAGCACCTCGCCGCTTACCTCCTCCTCGCCCTTGCTGGCAACACC
TCCCCGTCCTCTGAGGATGTCAAGGCCGTCCTCTCTTCCGTTGGCATT
GATGCCGATGAGGAGCGCCTGAACAAGCTCATTGCTGAGCTCGAGGGC
AAGGACCTCCAGGAGCTCATCGCCGAGGGTTCCACCAAGCTCGCTTCC
GTTCCCTCCGGTGGTGCTGCCGCCGCTGCTCCTGCCGCTGCCGGTGCC
GCTGCCGGTGGTGCTGCTGCTCCTGCCGCTGAGGAGAAGAAGGAGGAG
GAGAAGGAGGAGTCCGACGAGGACATGGGCTTCGGTCTTTTCGACTAA
SEQ ID NO 55:
Published P2 sequence for N.fischeri
gi|119480930|ref|1XM_001260493.1|Neosartorya
fischeri NRRL 181 60S acidic ribosomal protein
P2/allergen Asp F 8 (NFIA_085510) mRNA, complete
cds
ATGAAGCACCTCGCCGCTTACCTCCTCCTCGCCCTTGCTGGCAACACC
TCCCCGTCCGCTGAGGATGTCAAGGCCGTCCTCTCTTCCGTTGGCATT
GACGCCGATGAGGAGCGCCTGAACAAGCTCATTGCTGAGCTCGAGGGC
AAGGACCTCCAGGAGCTCATCGCCGAGGGTTCCACCAAGCTCGCTTCC
GTTCCCTCCGGTGGTGCTGCCGCCGCTGCTCCTGCCGCTGGCGGTGCC
GCTGCCGGTGGTGCTGCTGCTCCTGCCGCTGAGGAGAAGAAGGAGGAG
GAGAAGGAGGAGTCCGACGAGGACATGGGCTTCGGTCTCTTCGACTAA
SEQ ID NO 56:
Published P2 sequence for A.clavatus
gi|121715403|ref|XM_001275310.1| Aspergillus
clavatus NRRL 1 60S acidic ribosomal protein P2,
putative (ACLA_069130) mRNA, complete cds
ATGAAGCACCTCGCCGCTTACCTCCTCCTCGCCCTTGGTGGCAACGCC
AGCCCCTCCGCTGCTGATGTTAAGGAGGTTCTCTCTTCTGTCGGCATT
GATGCTGATGAGGAGCGCCTCAACAAGCTCATTGCTGAGCTCGAGGGC
AAGGACCTTCAGGAGCTGATTGCTGAGGGTTCCACCAAGCTCGCTTCC
ATTCCCTCCGGCGGTGCTGGTGGTGCTGCCCCCGCCGCTGGCGGTGCT
GCCGCCGGTGGTGCTGCTGAGGCCGCTCCCGCTGAGGAGAAGGAGGAG
GAGAAGGAGGAGTCCGACGACGACATGGGCTTCGGTCTCTTCGACTAA
SEQ ID NO 57:
Published P2 sequence for A.terreus gi|115395965|
ref|XM_001213622.1| Aspergillus terreus NIH2624
predicted protein (ATEG_04444) mRNA, complete cds
ATGAAGCACCTCGCCGCTTTCCTTCTCCTCGGCCTTGCCGGCAACACC
TCCCCCTCTGCTGAGGACATCAAGGCTGTCCTCTCCTCCGTCGGCATT
GACGCTGATGAGGAGCGCCTCGGCCAGCTCCTGAAGGAGCTCGAGGGC
AAGGACATCCAGGAGCTCATCGCTCAGGGCTCTGAGAAGCTCGCCTCC
GTTCCCTCTGGCGGTGCTGCCGCTGGTGCTGCTGCTCCCGCCGCTGCC
GCTGGCGGTGACGCTGCTGCCCCCGCTGAGAAGAAGGAGGAGGAGAAG
GAGGAGGAGTCCGACGAGGACATGGGCTTCGGTCTCTTCGACTAA
SEQ ID NO 58:
Published P2 sequence for A. niger gi|145252575|
ref|XM_001397764.1| Aspergillus niger CBS 513.88
hypothetical protein (An16g04930) mRNA, complete
cds
ATGAAGTACCTCGCCGCCTACCTTCTGTTGGCCCTTGCTGGCAACAAC
ACCCCCTCCGCTGAGGACATCAAGTCCGTCCTCTCCGCCGTCGGCATT
GACGCTGAGGAGGAGCGCCTCCAGAAGCTCCTTGCTGAGCTTGAGGGC
AAGGACCTCCAGGAGCTCATCTCCGAGGGTACCCAGAAGCTCGCTTCC
GTTCCCTCCGGTGGTGCCGGTGCTGCTGCCGCTGCCCCCGCTGCCGGT
GGCGCCGCTGCTGCTGAGGCTCCCGCTGAGGAGAAGAAGGAGGAGGCT
GCTGAGGAGTCCGATGAGGACATGGGCTTCGGTCTCTTCGACTAA
SEQ ID NO 59:
>gi|67539651|ref|XM_658508.1|Aspergillus
nidulans FGSC A4 chromosomel
ATGAAGCACCTCGCAGCCTACCTCCTCCTCGCCCTTGCTGGCAACGAG
TCTCCCTCCGCCTCCGACATCAAGGAGGTTCTCTCTTCCGTCGGTGTT
GACGCCGACGACGAGCGCCTCGAGAAGCTCATTGCTGAGCTCCAGGGC
AAGGACATCAACGAGCTGATCGCTGAGGGTACCACCAAGCTTGCCTCC
GTTCCCTCCGGCGGTGCTGGTGGTGCTGCCCCTGCTGCCGCTGCCGGT
GGTGCTGCTGCTGCCGAGGCCCCCGCTGCTGAGAAGGAGGAGGAGAAG
GAGGAGTCCGATGAGGACATGGGCTTCGGTCTCTTCGACTAA
SEQ ID NO 60:
>gi|11229039|gb|AF317661.1| Candida albicans
60S acidic ribosomal protein type P2-A (p2A)
gene, complete cds
GAATTCGATTATTGCATTCTGATATTCCCTGCTTTAAATGCATTTGGA
AATATTTCGTATATCATGAGATATAATAACATTAATAGCATTTTCATG
TTACTAACAAGAATATAGTGAAATACTTAGCTGCTTACTTATTATTAG
TTAACGCCGGTAACGCCACCCCATCTGCTGCCGATGTCAAAGCTGTTT
TGTCAGCTGCTGATATTGAAGTCGAAGAAGAAAAAGTTGAAAAATTGA
TCAGCGAATCGGACGGTAAGAACGTCGAAGAATTGATTGCTGAAGGTA
ACGAAAAATTATCATCAGTCCCATCTGGTGCTCCAGCTGCTGCTGCTG
GTGGTGCTTCTGCTGCCGCCGGTGGTGAAGCCACTGAAGAAGCTGCTG
AAGAAGAAGCTGCTGAAGAATCTGATGACGATATGAGTTTCGGTTTAT
TCGATTAAACGAGTCAACAGGCATCTCAAGATCACAGCATAAGG
A. fumigatus
SEQ ID NO 110:
>A. FUM505.62
TTACCTCCTCCTCGCCCTTGCTGGCAACACCTCCCCGTCCTCTGAGGA
TGTCAAGGCCGTCCTCTCTTCCGTTGGCATTGATGCCGATGAGGAGCG
CCTGAACAAGCTCATTGCTGAGCTCGAGGGCAAGGACCTCCAGGAGGT
TAGTAACTACAGCTCGAAGATTACAGACTGGGAATTTTGGACTGGCGC
TGACATCGAACTCTACAACAGCTCATTGCCGAGGGTTCCACCAAGCTC
GCTTCCGTTCCCTCCGGTGGTGCTGCCGCCGCTGCTCCTGCCGCTGCC
GGTGCCGCTGCCGGTGGTGCTGCTGCTCCTGCCGCTGAGGAGAAGAAG
GAGGAGGAGAAGGAGGAGTCCGACGAGGACATGGGCTTCGGTCT
A. flavus
SEQ ID NO 61:
>A.flavus2199\(P2)
TCGCCCTCGCTGGCAACTCCACCCCCTCCGTTGAGGACATCAAGAGCG
TTCTCTCTTCCGTCGGTATTGATGCCGATGAGGAGCGCCTCCAGAAGG
TCATCTCCGAGCTCGAGGGCAAGGACCTCCAGCAGGTTCGTAATACGT
TGAATGATTGAGACATAGGGCGCCTGCTGACCATCTACTCACTACAGC
TGATCACTGAGGGTAGCGAGAAGCTCGCTACCGTTCCCTCCGGTGGTG
CTGGTGCCGCTGCCCCTGCTGCTGGCGGTGCCGCTGCCGGTGGTGACG
CCCCCGCCGCTGAGGAGAAGGAGGAAGAGAAGGAGGAGTCCGATGAGG
ACATGGGCTTCGGTCT
A. niger
SEQ ID NO 62:
>A. nig2828\(P2F)
CTACCTTCTGTTGGCCCTTGCTGGCAACAACACCCCCTCCGCTGAGGA
CATCAAGTCCGTCCTCTCCGCCGTCGGCATTGACGCTGAGGAGGAGCG
CCTCCAGAAGCTCCTTGCTGAGCTTGAGGGCAAGGACCTCCAGGAGGT
CAGTTAACGCCCTTAAAAATCTACCAAGAAATTTTGCGATTACAAGAT
GGAATACTGACAATGGTTTTTTCTACAACAGCTCATCTCCGAGGGTAC
CCAGAAGCTCGCTTCCGTTCCCTCCGGTGGTGCCGGTGCTGCTGCCGC
TGCCCCCGCTGCCGGTGGCGCCGCTGCTGCTGAGGCTCCCGCTGAGGA
GAAGAAGGAGGAGGCTGCTGAGGAGTCCGATGAGGACATGGGCTTCGG
TCT
A. terreus
SEQ ID NO 63:
>A. terr307
TTTCCTTCTCCTCGGCCTTGCCGGCAACACCTCCCCCTCTGCTGAGGA
CATCAAGGCTGTCCTCTCCTCCGTCGGCATTGACGCTGATGAGGAGCG
CCTCGGCCAGCTCCTGAAGGAGCTCGAGGGCAAGGACATCCAGGAGGT
TAGTGATCACCATAACTTCGGACTACTGCGATGAAAACGCGCCATACT
AACGGATTATACAGCTCATCGCTCAGGGCTCTGAGAAGCTCGCCTCCG
TTCCCTCTGGCGGTGCTGCCGCTGGTGCTGCTGCTCCCGCCGCTGCCG
CTGGCGGTGACGCTGCTGCCCCCGCTGAGAAGAAGGAGGAGGAGAAGG
AGGAGGAGTCCGACGAGGACATGGGCTTCGGTCT
SEQ ID NO 64:
>A. terr5677
TTTCCTTCTCCTCGGCCTTGCCGGCAACACCTCCCCCTCTGCTGAGGA
CATCAAGGCTGTCCTCTCCTCCGTCGGCATTGACGCTGATGAGGAGCG
CCTCGGCCAGCTCCTGAAGGAGCTCGAGGGCAAGGACATCCAGGAGGT
TAGTGATCACCATAACTTCGGACTACTGCGATGAAAACGCGCCATACT
AACGGATTATACAGGTCATCGCTCAGGGCTCTGAGAAGCTCGCCTCCG
TTCCCTCTGGCGGTGCTGCCGCTGGTGCTGCTGCTCCCGCCGCTGCCG
CTGGCGGTGACGCTGCTGCCCCCGCTGAGAAGAAGGAGGAGGAGAAGG
AGGAGGAGTCCGACGAGGACATGGGCTTCGGTCT
SEQ ID NO 65:
>A. terr2729
TTTCCTTCTCCTCGGCCTTGCCGGCAACACCTCCCCCTCTGCTGAGGA
CATCAAGGCTGTCCTCTCCTCCGTCGGCATTGACGCTGATGAGGAGCG
CCTCGGCCAGCTCCTGAAGGAGCTCGAGGGCAAGGACATCCAGGAGGT
TAGTGATCACCATAACTTCGGACTACTGCGATGAAAACGCGCCATACT
AACGGATTATACAGCTCATCGCTCAGGGCTCTGAGAAGCTCGCCTCCG
TTCCCTCTGGCGGTGCTGCCGCTGGTGCTGCTGCTCCCGCCGCTGCCG
CTGGCGGTGACGCTGCTGCCCCCGCTGAGAAGAAGGAGGAGGAGAAGG
AGGAGGAGTCCGACGAGGACATGGGCTTCGGTCT
SEQ ID NO 66:
>A. terr601-65
TTTCCTTCTCCTCGGCCTTGCCGGCAACACCTCCCCCTCTGCTGAGGA
CATCAAGGCTGTCCTCTCCTCCGTCGGCATTGACGCTGATGAGGAGCG
CCTCGGCCAGCTCCTGAAGGAGCTCGAGGGCAAGGACATCCAGGAGGT
TAGTGATCACCATAACTTCGGACTACTGCGATGAAAACGCGCCATACT
AACGGATTATACAGCTCATCGCTCAGGGCTCTGAGAAGCTCGCCTCCG
TTCCCTCTGGCGGTGCTGCCGCTGGTGCTGCTGCTCCCGCCGCTGCCG
CTGGCGGTGACGCTGCTGCCCCCGCTGAGAAGAAGGAGGAGGAGAAGG
AGGAGGAGTCCGACGAGGACATGGGCTTCGGTCT
A. candidus
SEQ ID NO 67:
>A. Cand 225-8\(P2F)
TGCTCCTCGGCCTCGCMGGCAACGAGACTCCCTCCGCTGCCGACATCA
AGGGCGTTCTGTCCGCCGTCGGCATTGACGCCGATGAGGACCGTCTCT
CCAAGCTCCTCTCCGAGCTTGAGGGCAAGGACATCAACGAGGTTCGTA
TCTCACAGGAATCGCACACGTAACAGAGTCAACAAATACTAATCCCCC
GTGCAGCTGATCGCCCAGGGCTCCGAGAAGCTTGCTTCCGTTCCCTCC
GGTGGTGCCGCTGGTGGTGCCGCTGCCGCCCCTGCCGCCGCCGCTGGT
GGTGACGCTCCCGCCCAGGAGAAGGAGGAGGAGAAGGAGGAGTCCGAT
GAGGACATGGGCTTCGGTCT
SEQ ID NO 68:
>A. Cand14607\(P2F)
TGCTCCTCGGCCTCGCCGGCAACGAGTCTCCCTCCGCTGCCGACATCA
AGGGCGTTCTGTCCGCCGTCGGCATTGACGCCGATGAGGAGCGTCTCT
CCAAGCTCCTCTCCGAGCTTGAGGGCAAGGACATCAACGAGGTTCGTA
TCCTGGAATCGCACCGAGACAGAGTCAACAAATACTAATCCCCCGTGC
AGCTGATCGCCCAGGGTACCGAGAAGCTTGCTTCCGTTCCCTCCGGTG
GCGCCGGCGCTGCTGCTGCCGCCCCTGCCGCCGCTGCTGGTGGTGAGG
CCGCCGCTGAGGAGAAGAAGGAGGAGGAGAAGGAGGAGTCCGATGAGG
ACATGGGCTTCGGTCT
SEQ ID NO 69:
>A. Cand-9695\(APF)
GGCAACCGAGTCTCCCTCCGCTGCCGACATCAAGGGCGTTCTGTCCGC
CGTCGGCATTGACGCCGATGAGGAGCGTCTCTCCAAGCTCCTCTCCGA
GCTTGAGGGCAAGGACATCAACGAGGTTCGTATCCTGGAATCGCACCG
AGACAGAGTCAACAAATACTAATCCCCCGTGCAGCTGATCGCCCAGGG
TACCGAGAAGCTTGCTTCCGTTCCCTCCGGTGGCGCCGGCGCTGCTGC
TGCCGCCCCTGCCGCCGCTGCTGGTGGTGAGGCCGCCGCTGAGGAGAA
GAAGGAGGAGGAGAAGGAGGAGTCCGATGAGGACATGGGCTTCGGTCT
A. clavatus
SEQ ID NO 70:
>A. clav7944\(P2F)
TTACCTCCTCCTCGCCCTTGGTGGCAACGCCAGCCCCTCCGCTGCTGA
TGTTAAGGAGGTTCTCTCTTCCGTCGGCATTGATGCTGATGAGGAGCG
CCTCAACAAGCTCATTGCTGAGCTCGAGGGCAAGGACCTTCAGGAGGT
TAGTTTTACGCTGGTCTACGAGAGGAAGATTGTGACAAGATGCTAACG
GAAAATTTCTTCAACAGCTGATTGCTGAGGGTTCCACCAAGCTCGCTT
CCATTCCCTCCGGCGGTGCTGGTGGTGCTGCCCCCGCCGCTGGCGGTG
CTGCCGCCGGTGGTGCTGCTGAGGCCGCTCCCGCTGAGGAGAAGGAGG
AGGAGAAGGAGGAGTCCGACGACGACATGGGCTTCGGTCT
SEQ ID NO 71:
>A. clav443\(P2F)
TTACCTCCTCCTCGCCCTTGGTGGCAACGCCAGCCCCTCCGCTGCTGA
TGTTAAGGAGGTTCTCTCTTCCGTTGGCATTGATGCTGATGAGGAGCG
CCTCAATAAGCTCATTGCTGAGCTTGAGGGCAAGGACCTTCAGGAGGT
TAGTTTACGCCAATCCGCGATATGAAAATTGCGACATGATGCTAACAG
GAGAATTTTTCAACAGCTGATTGCTGAGGGTTCCACCAAGCTCGCTTC
CATTCCTTCCGGCGGTGCTGCCGGTGCTGCCCCTGCCGCTGGCGCTGC
TGCCGGTGGTGCCGCTGAGGCCGCTCCCGCTGAGGAGAAGGAGGAGGA
GAAGGAGGAGTCCGACGACGACATGGGCTTCGGTCT
A. glaucus
SEQ ID NO 72:
>A. glau2425\(P2F)
ACACGGTGGACTGGCTGGCCGGAAGAGCCGTTGCTGGTGTAAGTTTTG
GTAGGACCGTTCAGCTCCRGAKGTCTTTGGGGACAATGGCGACGAGGG
CTGTCGATCGTTAGTGTACATTCCAAGGAAGGTAAAAGAAAAAGAATG
CGACATACAGTAAGTTGAGCCACTCTGACGCTGGCAGTCATCGCAGTG
GTCATAAGCTGTAGCGAGCGGCTGCTCGATGTTGACCGAGTAGGTGAC
GGCTTTGCAGAGACAGTGACCGGAAATAGGCATTTTGGACGAGAGGCA
GAATATAATTGGACTATAGACTGACAATGATGATGACGATACTGAGGA
CCTTTGGGAGGAATGAAACAGATATTTATACCCTCTCCGACCTCGTGT
AGTCCATCTGTCTTTCGCTGTCTCTCGGTGGACATGGGCTTCGGTCT
SEQ ID NO 73:
>A. glau542
GCGTTGGTGCACGACGGTACCAAGGGGACTCTCATACTCCTTAATTCT
CTGGCCGGTCCAGTGTCGCGACTCRTATGGAGCACATTAGCAGATATC
CCACAGAGCTACCATGTCGTCCAGGCTCTTTGTCTGCTTTGCACATGG
CCTTTTCCAACCAGCAGTACCTCCACAGACCCGACGTTCATGCTGTGT
GGTATGATGATGCAAGTCGCCATGCAACTTGGTCTTCACCGGCCTTCG
CACACTCAGGACTTTASCAAGTTCACAGTGGAGCTGATTGAGGAGGAG
CTCAGGGATAAAGTGAGGACATGGGCTTCGGTCT
SEQ ID NO 74:
>A. glaucus29771\(P2)
GCGTTGGAGCACGACTGTACCAGGATACTCATCATCTCCTTAATTTTC
TGGCCGGTCCAGCGYCGCGACTAGTATGGAGCACATTATCAGACATCC
GACAGAGCTACCATGTCGTAAAGGCTCTTTGTCTGCTCTGCACATGGC
CTTTTCCGACCAGCAGTACCTCCACAGACCCTACKTTTATGCTGTGTG
GTATGATGATGCAAGTCGCCATGCAGCTTGGTCTTCACCGGCCTTCGC
ACACTCAGGACTTTAGCAAGTTTACAGTGGAGCTGATTGAGGAGGAAC
TAAGGGACAAAGTGAGGACATGGGCTTCGGTCT
A. versicolor
SEQ ID NO 75:
>A.Vers6898
CGCCCTTGCTGGCAACCAGAGCCCCTCTGCTTCCGATGTTAAGGAGGT
TCTCTCCTCCGTCGGTGTTGACGCTGACTCTGAGCGCCTCGAGAAGCT
CATCGCTGAGCTCCAGGGCAAGGACATCAACGAGGTTCGTTTTGACAG
AACCGCTTGAAATTCTTGGCAGCAGACTAATGAAACATTTTCTGCAGT
TGATCGCTGAGGGTACCACTAAGCTCGCTTCCGTTCCCTCTGGCGGTG
CTGGTGCTGCTGCTGCTCCCGCTGCTGGTGGCGCTGCTGCCGCTGAGG
CCCCTGCCGCCGAGAAGGAGGAGGAGAAGGAGGAGTCCGACGAGGACA
TGGGCTTCGGTCT
SEQ ID NO 76:
>lus66\(P2BF) sequence generated for C. lusitaniae
KTATTGTTGGKCAACGCTGGTAACACCGCCCCATCTGCTGCTGACGTC
AAGAAGGTCTTGGAATCCGTCTCTATTGAGGTTGAGGACGACAAGGTT
GAGAAGTTGTTGGCTGAAGTTGAAGGCAAGAACGTCGAAGAGTTGATT
GCCGAGGGTAACGAGAAGTTGTCTTCTGTTCCATCTGGTGCTCCAGCT
GCTGCTGGTGCCGCTGCTGCTTCTGGTTCTACTGAGGCTGCTGCTGAA
GAGCCACAAGAAGAAGAGAAGGAGGAGTCTGACGACGACATGGGTTTC
GGTTTATTCGATTA
SEQ ID NO 77:
>fam1\(P2BF) sequence generated for C. famata
CTTCTCCATCCGCTTCTGACATCAGTAGTTTATTAGAAACCGTTGGTG
CTGAAGCTGACGAAGCTAGAATCAGTGCTTTATTGAAGGACTTAGAAG
GTAAGCAAGTCGCTGACTTAATTGCTGAAGGTCAAACCAAGTTGGCTT
CCGTTCCAACTGGTGGTGCTGGTGCTGCTGCTGGTGGTGCCGCTGCTG
CTTCTGGTGATGCCGGTGCAGCTGCTGCTGAAGAAGAAAAGGAAGAAG
AAAAGGAAGAATCCGACGATGACATGGGTTTCGGTTTATTCGATTA
SEQ ID NO 78:
>fam2\(P2BF) sequence generated for C. famata
CTTCTCCATCAGCCTCTGACGTCAGTGCTTTATTAGAAACCGTTGGTG
CTGAAGTTGACCAAGGTAGAGTTAGTGCTTTATTGAAGGACTTAGAAG
GTAAGCAAGTTGCCGACTTAATTGCTGAAGGTCAAACCAAGTTAGCTT
CTGTCCCAACCGGTGGTGCTGCTTCTGCTGGTGGTGCTGCCGCTGCTT
CTGGTGCTGCCGGTGCAGCTGCTGTTGAAGAAGAAAAGGAAGAAGAAA
AGGAAGAATCCGATGAAGATATGGGTTTCGGTTTATTCGATT
SEQ ID NO 79:
>fam5\(P2BF) sequence generated for C. famata
CTTCTCCATCAGCCTCTGACGTCAGTGCTTTATTAGAAACCGTTGGTG
CTGAAGTTGACCAAGGTAGAGTCAGTGCTTTATTGAAGGACTTAGAAG
GTAAGCAAGTCGCCGACTTAATTGCTGAAGGTCAAACCAAGTTAGCTT
CTGTCCCAACTGGTGGTGCTGCTTCTGCTGGTGGTGCTTCCGCTGCTG
CTTCTGGTGATGCCGGTGCAGCTGCTGCTGAAGAAGAAAAGGAAGAAG
AAAAGGAAGAATCCGATGAAGATATGGGTTTCGGTTTATTCGATTA
SEQ ID NO 80:
>haem54\(P2BF) sequence generated for C. haemuloni
TTTCGGTTTATTCGATTAACACTTCCCCAGCTGCCTCTGACATCAAGA
AGGTGTTGGAGTCTGTCTCCATCGAGGTTGAGGACGACAAGGTCGAGA
AGTTGTTGGCTGAGGTCGAGGGCAAGAACGCCGAGGAGTTGATTGCCG
AGGGTAACGAGAAGTTGTCTTCTGTCCCAACTGGTGCTCCAGCTGGTG
GTGCTGCCGCTGCTGGTGGTGCTGCTCCAGAGGCTGCTGCTGAGAAGG
AAGAGGAGGCCGCTGCCGAGGAGTCTGACGACGACATGGGTTTCGGTT
TATTCGATTA
SEQ ID NO 81:
>haem55\(P2BF) sequence generated for C. haemuloni
TTATTGTTGGTCAACGCCGGTAACACTTCCCCAGCTGCCTCTGACATC
AAGAAGGTGTTGGAGTCTGTCTCCATCGAGGTTGAGGACGACAAGGTC
GAGAAGTTGTTGGCTGAGGTCGAGGGCAAGAACGCCGAGGAGTTGATT
GCCGAGGGTAACGAGAAGTTGTCTTCTGTCCCAACTGGTGCTCCAGCT
GGTGGTGCTGCCGCTGCTGGTGGTGCTGCTCCAGAGGCTGCTGCTGAG
AAGGAAGAGGAGGCCGCTGCCGAGGAGTCTGACGACGACATGGGTTTC
GGTTTATTCGATTA
SEQ ID NO 82:
>pul36\(P2BF) sequence generated for
C. pulcherrima
ACACCTCTCCATCCGCCGCCGATGTCAAGAAGGTCTTGGAGTCCGTTT
CCATCGAGGTTGAGGAGGACAAGGTCGAGAAGTTGCTCGCTGAGGTCG
AGGGCAAGAGCGTCGAGGACTTGATCGCTGAGGGTAACGAGAAGTTGT
CTTCTGTCCCAACTGGTGGCCCAGCCGCCGGTGGTGCCGCTGCCGCTG
CTGGTGGTGACGCCGCTCCTGCCGAGGAGGCCGCTGAGGAGGCCGCCG
AGGAGTCTGACGACGACATGGGTTTCGGTTTATTCGATTA
SEQ ID NO 83:
>pul39\(P2BF) sequence generated for
C. pulcherrima
CATCCGCCGCCGATGTCAAGRAGGTCTTGGAGTCCGTTTCCATCGAGG
TTGAGGAGGACAAGATCGAGAAGTTGCTCGCTGAGGTCGAGGGCAAGA
GCGTCGAGGACTTGATCGCTGAGGGTAACGAGAAGTTGTCTTCTGTCC
CAACTGGTGGCCCAGCCGCCGGTGGTGCCGCTGCCGCTGCCGGTGGTG
ACGCCGCTCCTGCCGAGGAGGCCGCTGAGGAGGCCGCCGAGGAGTCTG
ACGACGACATGGGTTTCGGTTTATTCGATTA
SEQ ID NO 84:
>U50\(P2BF) sequence generated for C. utilis
ACCGCCGACAAGATCACCTCCGTCTTGGAGTCTGTCGGTATTGAGGTT
GAGGAGTCCCAAGTCACCGAGTTGATCTCTGCCCTTGAGGGTAAGTCC
GTTGAGGAGCTCATTGCTGAAGGTAACGAGAAGTTGGCTTCTGTTCCA
ACCGGTGGTGCTGGTGCTGCTCCAGCTGCCGGTGCCGGTGCTGCTGAT
GCTGATGCTCCAGCTGAGGCTGCTGAGGAGGCTGCTGAGGAGGAGTCT
GACGATGACATGGGTTTCGGTTTATTCGATTA
Seq ID No 85
P2ForA
GAGGAGCGCCT
Seq ID No 86
P2ForB
GAGGAGCGCCTC
Seq ID No 87
P2ForC
GGAGGAGCGCCTC
Seq ID No 88
P2ForD
TGAGGAGCGCCTC
Seq ID No 89
P2RevA
CCGGAGGGAACGGA
Seq ID No 90
P2RevB
CCGGAGGGAACGG
SeqID No 91
P2FumP
ACTACAGCTCGAAGATTA
Seq ID No 92
P2FlavP
ACGTTGAATGATTGAGAC
Seq ID No 93
P2NigP
TTGCGATTACAAGATGGAA
Seq ID No 94
P2TerrP
CTTCGGACTACTGCGATGA
Seq ID No 95
CAP2BF (For)
ACCTCTCCATCAGCTTCTG
Seq ID No 96
CGP2BF (For)
ACCTCTGTCTTATCATCTGTCG
Seq ID No 97
CGP2BF (For)
AAGAAGGTTATCGAATCTGTTG
Seq ID No 98
CTP2BF (For)
TCCGCTTTATTGGAACAAGTTG
Seq ID No 99
CPP2BF (For)
TCCTCATTGTTGGAATCCGTTG
Seq ID 100
CAP2BR (Rev)
TCAGCAATCAATTCTTGC
SEQ ID 101
CKP2BR (Rev)
CTCTTCGACGGACTTACC
SEQ ID 102
CGP2BR (Rev)
TTCGTCCAAGGACTTACC
SEQ ID 103
CTP2BR (Rev)
TTCTTGCAAGTCTTTACC
SEQ ID 104
CPP2BR (Rev)
CTCGTTGATGTCTTTACC
SEQ ID 105
CAP2BP (P)
ACCGCTTTATTGGAATCCGTTG
SEQ ID 106
CKP2BP (P)
ATCCGACAAGTTAGACAAGTTAATC
SEQ ID 107
CGP2BP (P)
AGAATCAACGAATTGTTGTCTGC
SEQ ID 108
CTP2BP (P)
ATCTTCCAAATTAGACTTATTGTTGA
SEQ ID 109
CPP2BP (P)
GAAGAATCAAGATTATCTACCTTGTTG

### References

Abramczyk D, Tchórzewski M, Krokowski D, Boguszewska A, Grankowski N. Overexpression, purification and characterization of the acidic ribosomal P-proteins from Candida albicans. Biochim Biophys Acta. 2004 Jun. 11;1672(3):214-23.

Bailey-Serres J, Vangala S, Szick K, and Lee C H. Acidic phosphoprotein complex of the 60S ribosomal subunit of maize seedling roots. Components and changes in response to flooding. Plant Physiol. 1997 August; 114(4): 1293-1305.

Newton C H, Shimmin L C, Yee J, Dennis P P. (1990) A family of genes encode the multiple forms of the Saccharomyces cerevisiae ribosomal proteins equivalent to the Escherichia coli L12 protein and a single form of the L10-equivalent ribosomal protein. JBacteriol. 1990; 172: 579-588

Tchórzewski M, Krokowski D, Boguszewska A, Liljas A, Grankowski N. Structural characterization of yeast acidic ribosomal P proteins forming the P1A-P2B heterocomplex. Biochemistry. 2003 Apr. 1;42(12):3399-408.

Tchórzewski M, Boguszewska A, Dukowski P, Grankowski N. Oligomerization properties of the acidic ribosomal P-proteins from Saccharomyces cerevisiae: effect of P1A protein phosphorylation on the formation of the P1A-P2B hetero-complex. Biochim Biophys Acta. 2000 Dec 11;1499(1-2):63-73.

Wool I G, Chan Y L, Gluck A, Suzuki K The primary structure of rat ribosomal proteins PO, P1 and P2 and a proposal for a uniform nomenclature for mammalian and yeast ribosomal proteins. Biochimie 1991; 73: 861-870.

1. A diagnostic kit for a yeast or fungal species comprising an oligonucleotide probe capable of binding to at least a portion of the P2, P2A or P2B genes or its corresponding mRNA.

2. A kit as claimed in claim 1 wherein the portion of the P2 gene is selected from:
a portion of the region of the gene from base pair position 1 to base pair position 326 of the Aspergillus P2B gene,
a portion of base pair position 1 to base pair position 359 of the C. albicans P2B gene,
a portion of base pair position 114 to base pair position 439 of the C. albicans P2A gene, or
a portion of the region of the gene from base pair 24 to base pair position 158 in C. glabrata.

3. The kit of claim 1, comprising a probe for one or more of a portion of the region of the gene from base pair position 1 to base pair position 326 of the Aspergillus P2 gene, a portion of the region of the gene from base pair 24 to base pair position 158 in C. glabrata, a probe for a portion of base pair position 1 to base pair position 359 of the C. albicans P2B gene and a probe for a portion of base pair position 114 to by position 439 of the C. albicans P2A gene.

4. The kit of claim 1, wherein the probe is selected from SEQ ID NO 9, 10, 91-94, 105-110, or sequences substantially similar or complementary thereto which can also act as a probe.

5. The kit of claim 1, further comprising a primer for amplification of at least a portion of the P2, P2A and/or P2B gene.

6. The kit of claim 1, comprising a forward and a reverse primer for a portion of the P2, P2A and/or P2B gene.

7. The kit of claim 1, comprising at least one forward in vitro amplification primer and at least one reverse in vitro amplification primer, the forward amplification primer being selected from the group consisting of SEQ ID NO 1, 3, 7, 85-88, 95-99, or sequences substantially similar or complementary thereto which can also act as a forward amplification primer and the reverse amplification primer being selected from the group consisting of SEQ ID NO 2, 4, 5, 6, 8, 89, 90, 100-104 or sequences substantially similar or complementary thereto which can also act as a reverse amplification primer.

8. The kit of claim 1, based on direct nucleic acid detection technologies, signal amplification nucleic acid detection technologies, and nucleic acid in vitro amplification technologies is selected from one or more of Polymerase Chain Reaction (PCR), Ligase Chain Reaction (LCR), Nucleic Acids Sequence Based Amplification (NASBA), Strand Displacement Amplification (SDA), Transcription Mediated Amplification (TMA), Branched DNA technology (bDNA) and Rolling Circle Amplification Technology (RCAT) or other enzymatic in vitro amplification based technologies.

9. A nucleic acid molecule selected from the group consisting of: SEQ ID NO 1 through SEQ ID NO 110 and sequences substantially homologous or substantially complementary thereto or to a portion thereof and having a function in diagnostics based on the P2, P2A and/or P2B genes.

10. A nucleic acid molecule comprising an oligonucleotide having a sequence substantially homologous to or substantially complementary to a portion of a nucleic acid molecule as claimed in claim 9.

11. A method of detecting a target organism in a test sample comprising the steps of:
(i) Mixing the test sample with at least one oligonucleotide probe capable of binding to at least a portion of the P2, P2A and/or P2B gene or its corresponding mRNA under appropriate conditions;
(ii) hybridizing under a high stringency conditions any nucleic acid that may be present in the test sample with the oligonucleotide to form a probe:target duplex; and
(iii) determining whether a probe:target duplex is present; the presence of the duplex positively identifying the presence of the target organism in the test sample.

15. A method as claimed in claim 11 wherein the probe is selected from the group consisting of SEQ ID NO 9, 10, 91-94, 105-110 or sequences substantially homologous or substantially complementary thereto also capable of acting as a probe for the P2B gene.

16. The method of claim 11, wherein the target organism is a yeast and/or fungal species.

17. Use of a kit of claim 1 in a diagnostic assay to measure yeast and/or fungal titres in a patient.

18. A method of assessing the efficacy of a treatment regime designed to reduce yeast and/or fungal titre in a patient comprising use of a kit of claim 1, at one or more key stages of the treatment regime.

19. Use of a kit of claim 1, in a diagnostic assay to measure yeast and or fungal contamination in an environment.

20. Use as claimed in claim 19, wherein the environment is a hospital, a food sample, an environmental sample e.g. water, an industrial sample such as an in-process sample or an end product requiring bioburden or quality assessment.

21. Use of a kit of claim 1, in the identification and/or characterization of one or more disruptive agents that can be used to disrupt the P2, P2A or P2B gene function.

22. Use as claimed in claim 21, wherein the disruptive agent is selected from the group consisting of antisense RNA, PNA, siRNA.

23-25. (canceled)


SEQUENCE LISTING (110 sequences)

SEQ ID NO: 1 - DNA, 22 bp, Artificial Sequence
  Primer
attgttgacccaaggtggtaac

SEQ ID NO: 2 - DNA, 23 bp, Artificial Sequence
  Primer
tcgtccaaggacttaccttccaa

SEQ ID NO: 3 - DNA, 23 bp, Artificial Sequence
  Primer
tcgtccaaggacttaccttccaa

SEQ ID NO: 4 - DNA, 22 bp, Artificial Sequence
  Primer
taatcgaataaaccgaaaccca

SEQ ID NO: 5 - DNA, 18 bp, Artificial Sequence
  Primer
gcgacaagcaatttctct

SEQ ID NO: 6 - DNA, 22 bp, Artificial Sequence
  Primer
atcatcatcagattcttctttg

SEQ ID NO: 7 - DNA, 17 bp, Artificial Sequence
  Primer
atgaagcacctcgccgc

SEQ ID NO: 8 - DNA, 17 bp, Artificial Sequence
  Primer
agaccgaagcccatgtc

SEQ ID NO: 9 - DNA, 29 bp, Artificial Sequence
  Probe based on the P2B/P2 gene for C. glabrata and A. fumigatus.
caagaaggttatcgaatctgttggtattg

SEQ ID NO: 10 - DNA, 24 bp, Artificial Sequence
  Probe based on the P2B/P2 gene for C. glabrata and A. fumigatus.
cctgccgctgccggtgccgctgcc

SEQ ID NO: 11 - DNA, 308 bp, Candida glabrata
ctattgttgacccaaggtggtaacgaatctccagctgctgctgacatcaagaaggttatcgaatctgttggtattgaagctgacgaagccagaatcaacgaattgttgtctgctttggaaggtaagtccttggacgaattgatcgctgaaggtcaacaaaagttcgcctctgttccagttggtggtgctgctgctggtggtgcttccgctgctgctggtggtgccgctgccggtgaagccgctgaagaaaaggaagaagaagctgctgaagaatccgatgacgacatgggtttcggtttattcgatta

SEQ ID NO: 12 - DNA, 305 bp, Candida glabrata
ttgttgacccaaggtggtaacgaatctccagctgctgctgacatcaagaaggttatcgaatctgttggtattgaagctgacgaagccagaatcaacgaattgttgtctgctttggaaggtaagtccttggacgaattgatcgctgaaggtcaacaaaagttcgcctctgttccagttggtggtgctgctgctggtggtgcttccgctgctgctggtggtgccgctgccggtgaagccgctgaagaaaaggaagaagaagctgctgaagaatccgatgacgacatgggtttcggtttattcgatta

SEQ ID NO: 13 - DNA, 308 bp, Candida glabrata; identical to SEQ ID NO: 11

SEQ ID NO: 14 - DNA, 308 bp, Candida glabrata
ctattgttgacccaaggtggtaacgaatctccagctgctgctgacatcaagaaggttatcgaatctgttggtattgaagctgacgaagctagaatcaacgaattgttgtctgctttggaaggtaagtccttggacgaattgatcgctgaaggtcaacaaaagttcgcctctgttccagttggtggtgctgctgctggtggtgcttccgctgctgctggtggtgccgctgccggtgaagctgctgaagaaaaggaagaagaagctgctgaagaatccgatgacgacatgggtttcggtttattcgatta

SEQ ID NO: 15 - DNA, 296 bp, Candida krusei
ttactcttagtcaatgctggtaaaaccgcaccatctgctgcagatgttacctctgtcttatcatctgtcggtatcgaagttgaatccgacaagttagacaagttaatctccgaattagaaggtaagtccgtcgaagagttgattgctgaaggtactgaaaagatggcttctgctccaggtgcagcagctgctccagcttctggtgcaggtgcttccaccgaatctgctgcagcagaagaagttgaagaagaaaaggaagaatccgatgatgacatgggtttcggtttattcgatta

SEQ ID NO: 16 - DNA, 265 bp, Candida krusei
catctgctgcagatgttacctctgtcttatcatctgtcggtatcgaagttgaatccgacaagttagacaagttaatctccgaattagaaggtaagtccgtcgaagagttgattgctgaaggtactgaaaagatggcttctgctccaggtgcagcagctgctccagcttctggtgcaggtgcttccaccgaatctgctgcagcagaagaagttgaagaagaaaaggaagaatccgatgatgacatgggtttcggtttattcgatta

SEQ ID NO: 17 - DNA, 265 bp, Candida krusei; identical to SEQ ID NO: 16

SEQ ID NO: 18 - DNA, 296 bp, Candida krusei
ttactcttagtacatgctggtaaaaccgcaccatctgctgcagatgttacctctgtcttatcatctgtcggtatcgaagttgaatccgacaagttagacaagttaatctccgaattagaaggtaagtccgtcgaagagttgattgctgaaggtactgaaaagatggcttctgctccaggtgcagcagctgctccagcttctggtgcaggtgcttccaccgaatctgctgcagcagaagaagttgaagaagaaaaggaagaatccgatgatgacatgggtttcggtttattcgatta

SEQ ID NO: 19 - DNA, 331 bp, Candida albicans
ttattgttagttcaaggtggtaacacctctccatcagcttctgatatcaccgctttattggaatccgttggtgttgaagccgaagaatccagattacaagctttattgaaagatttggaaggtaaagacttgcaagaattgattgctgaaggtaacaccaaattagcttctgtcccatccggtggtgctgctgctggtggtgcttctgcctctgctggtgccgctgctggtggtgctgctgaagctgaagaagaaaaagaagaagaagccaaagaagaatctgatgatgatatgggtttcggtttattcgattagagaaattgcttgtcgc

SEQ ID NO: 20 - DNA, 331 bp, Candida albicans; identical to SEQ ID NO: 19

SEQ ID NO: 21 - DNA, 331 bp, Candida albicans; identical to SEQ ID NO: 19

SEQ ID NO: 22 - DNA, 331 bp, Candida albicans
ttattgttagttcaaggtggtaacacctctccatcagcttctgatatcaccgctttattggaatccgttggtgttgaagcygaagaatccagattacaagctttattgaaagatttggaaggtaaagacttgcaagaattgattgctgaaggtaacaccaaattagcttctgtcccatccggtggtgctgctgctggtggtgcttctgcctctrctggtgccgctgctggyggtgctgcygaagctgaagaagaaaaagaagaagaagccaaagaagaatctgatgatgatatgggtttcggtttattcgattagagaaattgcttgtcgc

SEQ ID NO: 23 - DNA, 331 bp, Candida dubliniensis
ttgttgttagttcaaggtggtaacgccactccatcagcttctgatatcagcgctgtcttggaaactgttggtgttgaagccgaagaatccagattacaagctttattgaaagatttggaaggtaaagatttgcaagaattgattgctgaaggtaacaccaaattagcttctgtcccaaccggtggtgctgctgctggtggtgcttccggttctgctggtgccgcttctggtgccgctgctgaagctgaagaagaaaaagaagaagaagctaaagaagaatctgatgatgatatgggtttcggtttattcgattagagaaattgcttgtcgc

SEQ ID NO: 24 - DNA, 292 bp, Candida dubliniensis
ttgttgttagttcaaggtggtaacgccactccatcagcttctgatatcagcgctgtcttggaaactgttggtgttgaagccgaagaatccagattacaagctttattgaaagatttggaaggtaaagatttgcaagaattgattgctgaaggtaacaccaaattagcttctgtcccaaccggtggtgctgctgctggtggtgcttccggttctgctggtgccgcttctggtgccgctgctgaagctgaagaagaaaaagaagaagaagccaaagaagaatctgatgatgata

SEQ ID NO: 25 - DNA, 338 bp, Candida parapsilosis
atgaaatacttagctgcttacttattattggtccaaggtggtaacgcctccccatctgcttcagacatctcctcattgttggaatccgttggtgttgaagttgaagaatcaagattatctaccttgttgaaagacttggaaggtaaagacatcaacgagttgattgctgaaggtaacaccaaattggcctcagttccatctggtggtgctgctgttgcttccggttctggtgcttctggtgccgctgctggtggtgctgctgaagaagctaaggaagaagccaaggaagaagaaaaggaagaatctgatgatgacatgggtttcggtttattcgatta

SEQ ID NO: 26 - DNA, 317 bp, Candida parapsilosis
ttattattggtccaaggtggtaacgcctccccatctgcttcagacatctcctcattgttggaatccgttggtgttgaagttgaagaatcaagattatctaccttgttgaaagacttggaaggtaaagacatcaacgagttgattgctgaaggtaacaccaaattggcctcagttccatctggtggtgctgctgttgcttccggttctggtgcttctggtgccgctgctggtggtgctgctgaagaagctaaggaagaagccaaggaagaagaaaaggaagaatctgatgatgacatgggtttcggtttattcgatta

SEQ ID NO: 27 - DNA, 255 bp, Candida parapsilosis
ggtggtaacgcctccccatccgcttcagacatctcatccttgttggaatccgttggtgttgaagttgaagaatcaagattgtccctcttgttgaaagacttggaaggtaaagacatcaacgaattgattgctgaaggtaacaccaagttggcttcagttccaactggtggtgctgctgttgcttctggttctggtgcttcaggtgccgctgctggtggtgctgctgaagaagccaaagaagaatctgatgatgat

SEQ ID NO: 28 - DNA, 273 bp, Candida tropicalis
ggtggtaacgcttccccatctgcttctgacatctccgctttattggaacaagttggtgctgaagttgaatcttccaaattagacttattgttgaaagaattggaaggtaaagacttgcaagaattgattgccgaaggtaacactaaattcgcctctgtcccatccggtggtgctgctgctgcttcytctggttccgctgccgctgctggtggtgccgctgccgaagctgaagaagaaaaagaagaagaagccaaagaagaatctgatgatgat

SEQ ID NO: 29 - DNA, 273 bp, Candida tropicalis
ggtggtaacgcttccccatctgcttctgacatctccgctttattggaacaagttggtgctgaagttgaatcttccaaattagacttattgttgaaagaattggaaggtaaagacttgcaagaattgattgccgaaggtaacactaaattcgcctctgtcccatccggtggtgctgctgctgcttcctctggttccgctgccgctgctggtggtgccgctgccgaagctgaagaagaaaaagaagaagaagccaaagaagaatctgatgatgat

SEQ ID NO: 30 - DNA, 288 bp, Candida tropicalis
ttattattagtccaaggtggtaacrcttccccatctgcttctgacatctccgctttattggaacaagttggtgctgaagttgaatcttccaaattagacttattgttgaaagaattggaaggtaaagacttgcaagaattgattgccgaaggtaacactaaattcgcctctgtcccatccggtggtgctgcygctgcttcctctggttccgctgccgctgctggtggtgccgctgccgaagctgaagaagaaaaagaagaagaagccaaagaagaatctgatgatgat

SEQ ID NO: 31 - DNA, 288 bp, Candida tropicalis
ttattattagtccaaggtggtaacgcttccccatctgcttctgacatctccgctttattggaacaagttggtgctgaagttgaatcttccaaattagacttattgttgaaagaattggaaggtaaagacttgcaagaattgattgccgaaggtaacactaaattcgcctctgtcccatccggtggtgctgctgctgcttcctctggttccgctgccgctgctggtggtgccgctgccgaagctgaagaagaaaaagaagaagaagccaaagaagaatctgatgatgat

SEQ ID NO: 32 - DNA, 288 bp, Candida tropicalis
ttattattagtccaaggtggtaacgcttccccatctgcttctgacatctccgctttattggaacaagttggtgctgaagttgaatcttccaaattagacttattgttgaaagaattggaaggtaaagacttgcaagaattgattgccgaaggtaacactaaattcgcctctgtcccatccggtggtgctgctgctgcttcttctggttccgctgccgctgctggtggtgccgctgccgaagctgaagaagaaaaagaagaagaagccaaagaagaatctgatgatgat

SEQ ID NO: 33 - DNA, 302 bp, Candida guilliermondii
ttrttgttggtkracgccggtaacacctccccatctgctgctgacatcaaggctgtcttggagtcggtttccatagaagttgacgacgagaaggtgtccaagttgttgagcgaagttgagggaaagaatgctgaagaattgatcgctgaaggtaacgaaaaattgtcttctgttccaactggtggaccagctgctgcttcctctggatctgctgccgctgccgatgctcctgctgccgaagaggccgctgaggaggccgctgaggagtctgacgacgacatgggtttcggtttattcgatta

SEQ ID NO: 34 - DNA, 302 bp, Candida guilliermondii; identical to SEQ ID NO: 33

SEQ ID NO: 35 - DNA, 302 bp, Candida lusitanie
ttattgttggtcaacgctggtaacaccgccccatctgctgctgacgtcaagaaggtcttggaatccgtctctattgaggttgaggacgacaaggttgagaagttgttggctgaagttgaaggcaagaacgtcgaagagttgattgccgagggtaacgagaagttgtcttctgttccatctggtgctccagctgctgctggtgccgctgctgcttctggttctactgaggctgctgctgaagagccacaagaagaagagaaggaggagtctgacgacgacatgggtttcggtttattcgatta

SEQ ID NO: 36 - DNA, 380 bp, Aspergillus fumigatus
ttacctcctcctcgcccttgctggcaacacctccccgtcctctgaggatgtcaaggccgtcctctcttccgttggcattgatgccgatgaggagcgcctgaacaagctcattgctgagctcgagggcaaggacctccaggaggttagtaactacagctcgaagattacagactgggaattttggactggcgctgacatcgaactctacaacagctcattgccgagggttccaccaagctcgcttccgttccctccggtggtgctgccgccgctgctcctgccgctgccggtgccgctgccggtggtgctgctgctcctgccgctgaggagaagaaggaggaggagaaggaggagtccgacgaggacatgggcttcggtct

SEQ ID NO: 37 - DNA, 377 bp, Aspergillus fumigatus
cctcctcctcgcccttgctggcaacacctccccgtcctctgaggatgtcaaggccgtcctctcttccgttggcattgatgccgatgaggagcgcctgaacaagctcattgctgagctcgagggcaaggacctccaggaggttagtaactacagctcgaagattacagactgggaattttggactggcgctgacatcgaactctacaacagctcatcgccgagggttccaccaagctcgcttccgttccctccggtggtgctgccgccgctgctcctgccgctgccggtgccgctgccggtggtgctgctgctcctgccgctgaggagaagaaggaggaggagaaggaggagtccgacgaggacatgggcttcggtct

SEQ ID NO: 38 - DNA, 357 bp, Aspergillus fumigatus
gcaacacctccccgtcctctgaggatgtcaaggccgtcctctcttccgttggcattgatgccgatgaggagcgcctgaacaagctcattgctgagctcgagggcaaggacctccaggaggttagtaactacagctcgaagattacagactgggaattttggactggcgctgacatcgaactctacaacagctcattgccgagggttccaccaagctcgcttccgttccctccggtggtgctgccgccgctgctcctgccgctgccggtgccgctgccggtggtgctgctgctcctgccgctgaggagaagaaggaggaggagaaggaggagtccgacgaggacatgggcttcggtct

SEQ ID NO: 39 - DNA, 380 bp, Aspergillus fumigatus; identical to SEQ ID NO: 36

SEQ ID NO: 40 - DNA, 380 bp, Neosartorya fischeri
ttacctcctcctcgcccttgctggcaacacctccccctccgctgaggatgtcaaggccgtcctctcttccgtcggcattgacgccgatgaggagcgcctgaacaagctcattgctgagctcgagggcaaggacctccaggaggttagtacacacggcttgaatattaccgactgagaattttggaccggcgctgacatcgatttctacaacagctgatcgctgagggttccgccaagctcgcttccgttccctccggtggtgccggtggtgccgctgctcctgccgctggcggtgccgctgccggtggtgctgctgccgctcccgccgaagagaaggaggaggagaaggaggagtccgacgaggacatgggcttcggtct

SEQ ID NO: 41 - DNA, 365 bp, Aspergillus nidulans
ctacctcctcctcgcccttgctggcaacgagtctccctccgcctccgacatcaaggaggttctctcttccgtcggtgttgacgccgacgacgagcgcctcgagaagctcattgctgagctccagggcaaggacatcaacgaggttcgttattgcatatagagttggaagacgcggactgcgggctaacgataatctttaaacagctgatcgctgagggtaccaccaagcttgcctccgttccctccggcggtgctggtggtgctgcccctgctgccgctgccggtggtgctgctgctgccgaggcccccgctgctgagaaggaggaggagaaggaggagtccgatgaggacatgggcttcggtct

SEQ ID NO: 42 - DNA, 349 bp, Aspergillus nidulans
ccttgctggcaacgagtctccctccgcctccgacatcaaggaggttctctcttccgtcggtgttgacgccgacgacgagcgcctcgagaagctcattgctgagctccagggcaaggacatcaacgaggttcgttattgcatagggttggaagacgcggacagcgggctaacgataatcttctgaacagctgatcgctgagggtaccaccaagcttgcctccgttcccaccggcggtgctggtgctgctgcccctgctgccgctgccggtggtgctgccgctgccgaggctcccgctgctgagaaggaggaggagaaggaggagtccgatgaggacatgggcttcggtct

SEQ ID NO: 43 - DNA, 376 bp, Aspergillus clavatus
ttacctcctcctcgcccttggtggcaacgccagcccctccgctgctgatgttaaggaggttctctcttctgtcggcattgatgctgatgaggagcgcctcaacaagctcattgctgagctcgagggcaaggaccttcaggaggttagttttgcgctggtctacgagaggaagattgtgacaagatgctaacggaaaatttcttcaacagctgattgctgagggttccaccaagctcgcttccattccctccggcggtgctggtggtgctgcccccgccgctggcggtgctgccgccggtggtgctgctgaggccgctcccgctgaggagaaggaggaggagaaggaggagtccgacgacgacatgggcttcggtct

SEQ ID NO: 44 - DNA, 385 bp, Aspergillus niger
ctaccttctgttggcccttgctggcaacaacaccccctccgctgaggacatcaagtccgtcctctccgccgtcggcattgacgctgaggaggagcgcctccagaagctccttgctgagcttgagggcaaggacctccaggaggtcagttaacgcccttaaaatctaccaagaaattttgcgattacaagatggaatactgacaatgggttttctacaacagctcatctccgagggtacccagaagctcgcttccgttccctccggtggtgccggtgctgctgccgctgcccccgctgccggtggcgccgctgctgctgaggctcccgctgaggagaagaaggaggaggctgctgaggagtccgatgaggacatgggcttcggtct

SEQ ID NO: 45 - DNA, 385 bp, Aspergillus niger
ctaccttctgttggcccttgctggcaacaacaccccctccgctgaggacatcaagtccgtcctctccgccgtcggcattgacgctgaggaggagcgcctccagaagctccttgctgagcttgagggcaaggacctccaggaggtcagttaacgcccttaaaatctaccaagaaattttgcgattacaagatggaatactgacaatggtttttctacaacagctcatctccgagggtacccagaagctcgcttccgttccctccggtggtgccggtgctgctgccgctgcccccgctgccggtggcgccgctgctgctgaggctcccgctgaggagaagaaggaggaggctgctgaggagtccgatgaggacatgggcttcggtct

SEQ ID NO: 46 - DNA, 366 bp, Aspergillus terreus
ttccttctcctcggccttgccggcaacacctccccctctgctgaggacatcaaggctgtcctctcctccgtcggcattgacgctgatgaggagcgcctcggccagctcctgaaggagctcgagggcaaggacatccaggaggttagtgatcaccataacttcggactactgcgatgagaacgcgccatactaacggattatacagctcatcgctcagggctctgagaagctcgcttccgttccctctggcggtgctgccgctgctgctgctcccgccgctgccgctggcggtgacgctgctgcccccgctgagaagaaggaggaggagaaggaggaggagtccgacgaggacatgggcttcggtct

SEQ ID NO: 47 - DNA, 363 bp, Aspergillus flavus
ttacctcctcctcgccctcgctggcaactccaccccctccgttgaggacatcaagagcgttctctcttccgtcggtattgatgccgatgaggagcgcctccagaaggtcatctccgagctcgagggcaaggacctccagcaggttcgtaatacgttgaatgattgagacatagggcgcctgctgaccatctactcactacagctgatcactgagggtagcgagaagctcgctaccgttccctccggtggtgctggtgccgctgcccctgctgctggcggtgccgctgccggtggtgacgcccccgccgctgaggagaaggaggaagagaaggaggagtccgatgaggacatgggcttcggtct

SEQ ID NO: 48 - DNA, 351 bp, Aspergillus versicolor
tcgcccttgctggcaacgagagcccctctgcttccgacattaaggaggttctgtcctccgtcggtgttgacgctgacaacgagcgcctcgagaagctcatcgctgagctccagggcaaggacatcaacgaggttcgttttgacagatgcatttgaaatacttggccagcagactaatgaaacctcttctgcagttgatcgctgagggtaccaccaagctcgcttccgttccctctggcggtggtggtggtgctgccgcccccgctgctggtggcgctgccgccgctgaggcccctgctgctgagaaggaggaggagaaggaggagtccgacgaggacatgggcttcggtct

SEQ ID NO: 49 - DNA, 348 bp, Aspergillus versicolor
cccttgctggcaacgagagcccctctgcttccgacattaaggaggttctgtcctccgtcggtgttgacgctgacgacgagcgcctcgagaagctcatcgctgagctccagggcaaggacatcaacgaggttcgttttgacagatgcgtttgaaatacttggccagcagactaatgaaacctcttctgcagttgatcgctgagggtaccaccaagctcgcttccgttccctctggcggtggtggtggtgctgccgcccccgctgctggtggcgctgccgccgctgaggcccctgctgctgagaaggaggaggagaaggaggagtccgacgaggacatgggcttcggtct

SEQ ID NO: 50 - DNA, 481 bp, Candida albicans
ggaaaaaatgaaatacttagctgcttacttattgttagttcaaggtggtaacacctctccatcagcttctgatatcaccgctttattggaatccgttggtgttgaagccgaagaatccagattacaagctttattgaaagatttggaaggtaaagacttgcaagaattgattgctgaaggtaacaccaaattagcttctgtcccatccggtggtgctgctgctggtggtgcttctgcctctactggtgccgctgctggtggtgctgccgaagctgaagaagaaaaagaagaagaagccaaagaagaatctgatgatgatatgggtttcggtttattcgattagagaaattgcttgtcgcctttgctggtttgagagaagtatatttccattattttgcattatatatatatatatgtattataactaatctaataaaaaaaatatgaaaacaaaaatggcttctatatggcactgtttgca

SEQ ID NO: 51 - DNA, 330 bp, Candida glabrata
atgaagtacttggccgcttacctattgttgacccaaggtggtaacgaatctccagctgctgctgacatcaagaaggttatcgaatctgttggtattgaagctgacgaagctagaatcaacgaattgttgtctgctttggaaggtaagtccttggacgaattgatcgctgaaggtcaacaaaagttcgcctctgttccagttggtggtgctgctgctggtggtgcttccgctgctgctggtggtgccgctgccggtgaagctgctgaagaaaaggaagaagaagctgctgaagaatccgatgacgacatgggtttcggtttgttcgactaa

SEQ ID NO: 52 - DNA, 336 bp, Candida albicans
atgaaatacttagctgcttacttattgttagttcaaggtggtaacacctctccatcagcttctgatatcaccgctttattggaatccgttggtgttgaagccgaagaatccagattacaagctttattgaaagatttggaaggtaaagacttgcaagaattgattgctgaaggtaacaccaaattagcttctgtcccatccggtggtgctgctgctggtggtgcttctgcctctgctggtgccgctgctggtggtgctgctgaagctgaagaagaaaaagaagaagaagccaaagaagaatctgatgatgatatgggtttcggtttattcgattag

SEQ ID NO: 53 - DNA, 336 bp, Candida albicans; identical to SEQ ID NO: 52

SEQ ID NO: 54 - DNA, 336 bp, Aspergillus fumigatus
atgaagcacctcgccgcttacctcctcctcgcccttgctggcaacacctccccgtcctctgaggatgtcaaggccgtcctctcttccgttggcattgatgccgatgaggagcgcctgaacaagctcattgctgagctcgagggcaaggacctccaggagctcatcgccgagggttccaccaagctcgcttccgttccctccggtggtgctgccgccgctgctcctgccgctgccggtgccgctgccggtggtgctgctgctcctgccgctgaggagaagaaggaggaggagaaggaggagtccgacgaggacatgggcttcggtcttttcgactaa

SEQ ID NO: 55 - DNA, 336 bp, Neosartorya fischeri
atgaagcacctcgccgcttacctcctcctcgcccttgctggcaacacctccccgtccgctgaggatgtcaaggccgtcctctcttccgttggcattgacgccgatgaggagcgcctgaacaagctcattgctgagctcgagggcaaggacctccaggagctcatcgccgagggttccaccaagctcgcttccgttccctccggtggtgctgccgccgctgctcctgccgctggcggtgccgctgccggtggtgctgctgctcctgccgctgaggagaagaaggaggaggagaaggaggagtccgacgaggacatgggcttcggtctcttcgactaa

SEQ ID NO: 56 - DNA, 336 bp, Aspergillus clavatus
atgaagcacctcgccgcttacctcctcctcgcccttggtggcaacgccagcccctccgctgctgatgttaaggaggttctctcttctgtcggcattgatgctgatgaggagcgcctcaacaagctcattgctgagctcgagggcaaggaccttcaggagctgattgctgagggttccaccaagctcgcttccattccctccggcggtgctggtggtgctgcccccgccgctggcggtgctgccgccggtggtgctgctgaggccgctcccgctgaggagaaggaggaggagaaggaggagtccgacgacgacatgggcttcggtctcttcgactaa

SEQ ID NO: 57 - DNA, 333 bp, Aspergillus terreus
atgaagcacctcgccgctttccttctcctcggccttgccggcaacacctccccctctgctgaggacatcaaggctgtcctctcctccgtcggcattgacgctgatgaggagcgcctcggccagctcctgaaggagctcgagggcaaggacatccaggagctcatcgctcagggctctgagaagctcgcctccgttccctctggcggtgctgccgctggtgctgctgctcccgccgctgccgctggcggtgacgctgctgcccccgctgagaagaaggaggaggagaaggaggaggagtccgacgaggacatgggcttcggtctcttcgactaa

SEQ ID NO: 58 - DNA, 333 bp, Aspergillus niger
atgaagtacctcgccgcctaccttctgttggcccttgctggcaacaacaccccctccgctgaggacatcaagtccgtcctctccgccgtcggcattgacgctgaggaggagcgcctccagaagctccttgctgagcttgagggcaaggacctccaggagctcatctccgagggtacccagaagctcgcttccgttccctccggtggtgccggtgctgctgccgctgcccccgctgccggtggcgccgctgctgctgaggctcccgctgaggagaagaaggaggaggctgctgaggagtccgatgaggacatgggcttcggtctcttcgactaa

SEQ ID NO: 59 - DNA, 330 bp, Aspergillus nidulans
atgaagcacctcgcagcctacctcctcctcgcccttgctggcaacgagtctccctccgcctccgacatcaaggaggttctctcttccgtcggtgttgacgccgacgacgagcgcctcgagaagctcattgctgagctccagggcaaggacatcaacgagctgatcgctgagggtaccaccaagcttgcctccgttccctccggcggtgctggtggtgctgcccctgctgccgctgccggtggtgctgctgctgccgaggcccccgctgctgagaaggaggaggagaaggaggagtccgatgaggacatgggcttcggtctcttcgactaa

SEQ ID NO: 60 - DNA, 476 bp, Candida albicans
gaattcgattattgcattctgatattccctgctttaaatgcatttggaaatatttcgtatatcatgagatataataacattaatagcattttcatgttactaacaagaatatagtgaaatacttagctgcttacttattattagttaacgccggtaacgccaccccatctgctgccgatgtcaaagctgttttgtcagctgctgatattgaagtcgaagaagaaaaagttgaaaaattgatcagcgaatcggacggtaagaacgtcgaagaattgattgctgaaggtaacgaaaaattatcatcagtcccatctggtgctccagctgctgctgctggtggtgcttctgctgccgccggtggtgaagccactgaagaagctgctgaagaagaagctgctgaagaatctgatgacgatatgagtttcggtttattcgattaaacgagtcaacaggcatctcaagatcacagcataagg

SEQ ID NO: 61 - DNA, 352 bp, Aspergillus flavus
tcgccctcgctggcaactccaccccctccgttgaggacatcaagagcgttctctcttccgtcggtattgatgccgatgaggagcgcctccagaaggtcatctccgagctcgagggcaaggacctccagcaggttcgtaatacgttgaatgattgagacatagggcgcctgctgaccatctactcactacagctgatcactgagggtagcgagaagctcgctaccgttccctccggtggtgctggtgccgctgcccctgctgctggcggtgccgctgccggtggtgacgcccccgccgctgaggagaaggaggaagagaaggaggagtccgatgaggacatgggcttcggtct

SEQ ID NO: 62 - DNA, 386 bp, Aspergillus niger
ctaccttctgttggcccttgctggcaacaacaccccctccgctgaggacatcaagtccgtcctctccgccgtcggcattgacgctgaggaggagcgcctccagaagctccttgctgagcttgagggcaaggacctccaggaggtcagttaacgcccttaaaaatctaccaagaaattttgcgattacaagatggaatactgacaatggtttttctacaacagctcatctccgagggtacccagaagctcgcttccgttccctccggtggtgccggtgctgctgccgctgcccccgctgccggtggcgccgctgctgctgaggctcccgctgaggagaagaaggaggaggctgctgaggagtccgatgaggacatgggcttcggtct

SEQ ID NO: 63 - DNA, 370 bp, Aspergillus terreus
tttccttctcctcggccttgccggcaacacctccccctctgctgaggacatcaaggctgtcctctcctccgtcggcattgacgctgatgaggagcgcctcggccagctcctgaaggagctcgagggcaaggacatccaggaggttagtgatcaccataacttcggactactgcgatgaaaacgcgccatactaacggattatacagctcatcgctcagggctctgagaagctcgcctccgttccctctggcggtgctgccgctggtgctgctgctcccgccgctgccgctggcggtgacgctgctgcccccgctgagaagaaggaggaggagaaggaggaggagtccgacgaggacatgggcttcggtct

SEQ ID NO: 64 - DNA, 370 bp, Aspergillus terreus
tttccttctcctcggccttgccggcaacacctccccctctgctgaggacatcaaggctgtcctctcctccgtcggcattgacgctgatgaggagcgcctcggccagctcctgaaggagctcgagggcaaggacatccaggaggttagtgatcaccataacttcggactactgcgatgaaaacgcgccatactaacggattatacaggtcatcgctcagggctctgagaagctcgcctccgttccctctggcggtgctgccgctggtgctgctgctcccgccgctgccgctggcggtgacgctgctgcccccgctgagaagaaggaggaggagaaggaggaggagtccgacgaggacatgggcttcggtct

SEQ ID NO: 65 - DNA, 370 bp, Aspergillus terreus; identical to SEQ ID NO: 63

SEQ ID NO: 66 - DNA, 370 bp, Aspergillus terreus; identical to SEQ ID NO: 63

SEQ ID NO: 67 - DNA, 356 bp, Aspergillus candidus
tgctcctcggcctcgcmggcaacgagactccctccgctgccgacatcaagggcgttctgtccgccgtcggcattgacgccgatgaggaccgtctctccaagctcctctccgagcttgagggcaaggacatcaacgaggttcgtatctcacaggaatcgcacacgtaacagagtcaacaaatactaatcccccgtgcagctgatcgcccagggctccgagaagcttgcttccgttccctccggtggtgccgctggtggtgccgctgccgcccctgccgccgccgctggtggtgacgctcccgcccaggagaaggaggaggagaaggaggagtccgatgaggacatgggcttcggtct

SEQ ID NO: 68 - DNA, 352 bp, Aspergillus candidus
tgctcctcggcctcgccggcaacgagtctccctccgctgccgacatcaagggcgttctgtccgccgtcggcattgacgccgatgaggagcgtctctccaagctcctctccgagcttgagggcaaggacatcaacgaggttcgtatcctggaatcgcaccgagacagagtcaacaaatactaatcccccgtgcagctgatcgcccagggtaccgagaagcttgcttccgttccctccggtggcgccggcgctgctgctgccgcccctgccgccgctgctggtggtgaggccgccgctgaggagaagaaggaggaggagaaggaggagtccgatgaggacatgggcttcggtct

SEQ ID NO: 69 - DNA, 336 bp, Aspergillus candidus
ggcaaccgagtctccctccgctgccgacatcaagggcgttctgtccgccgtcggcattgacgccgatgaggagcgtctctccaagctcctctccgagcttgagggcaaggacatcaacgaggttcgtatcctggaatcgcaccgagacagagtcaacaaatactaatcccccgtgcagctgatcgcccagggtaccgagaagcttgcttccgttccctccggtggcgccggcgctgctgctgccgcccctgccgccgctgctggtggtgaggccgccgctgaggagaagaaggaggaggagaaggaggagtccgatgaggacatgggcttcggtct

SEQ ID NO: 70 - DNA, 376 bp, Aspergillus clavatus
ttacctcctcctcgcccttggtggcaacgccagcccctccgctgctgatgttaaggaggttctctcttccgtcggcattgatgctgatgaggagcgcctcaacaagctcattgctgagctcgagggcaaggaccttcaggaggttagttttacgctggtctacgagaggaagattgtgacaagatgctaacggaaaatttcttcaacagctgattgctgagggttccaccaagctcgcttccattccctccggcggtgctggtggtgctgcccccgccgctggcggtgctgccgccggtggtgctgctgaggccgctcccgctgaggagaaggaggaggagaaggaggagtccgacgacgacatgggcttcggtct

SEQ ID NO: 71 - DNA, 372 bp, Aspergillus clavatus
ttacctcctcctcgcccttggtggcaacgccagcccctccgctgctgatgttaaggaggttctctcttccgttggcattgatgctgatgaggagcgcctcaataagctcattgctgagcttgagggcaaggaccttcaggaggttagtttacgccaatccgcgatatgaaaattgcgacatgatgctaacaggagaatttttcaacagctgattgctgagggttccaccaagctcgcttccattccttccggcggtgctgccggtgctgcccctgccgctggcgctgctgccggtggtgccgctgaggccgctcccgctgaggagaaggaggaggagaaggaggagtccgacgacgacatgggcttcggtct

SEQ ID NO: 72 - DNA, 431 bp, Aspergillus glaucus
acacggtggactggctggccggaagagccgttgctggtgtaagttttggtaggaccgttcagctccrgakgtctttggggacaatggcgacgagggctgtcgatcgttagtgtacattccaaggaaggtaaaagaaaaagaatgcgacatacagtaagttgagccactctgacgctggcagtcatcgcagtggtcataagctgtagcgagcggctgctcgatgttgaccgagtaggtgacggctttgcagagacagtgaccggaaataggcattttggacgagaggcagaatataattggactatagactgacaatgatgatgacgatactgaggacctttgggaggaatgaaacagatatttataccctctccgacctcgtgtagtccatctgtctttcgctgtctctcggtggacatgggcttcggtct

SEQ ID NO: 73 - DNA, 322 bp, Aspergillus glaucus
gcgttggtgcacgacggtaccaaggggactctcatactccttaattctctggccggtccagtgtcgcgactcrtatggagcacattagcagatatcccacagagctaccatgtcgtccaggctctttgtctgctttgcacatggccttttccaaccagcagtacctccacagacccgacgttcatgctgtgtggtatgatgatgcaagtcgccatgcaacttggtcttcaccggccttcgcacactcaggactttascaagttcacagtggagctgattgaggaggagctcagggataaagtgaggacatgggcttcggtct

SEQ ID NO: 74 - DNA, 321 bp, Aspergillus glaucus
gcgttggagcacgactgtaccaggatactcatcatctccttaattttctggccggtccagcgycgcgactagtatggagcacattatcagacatccgacagagctaccatgtcgtaaaggctctttgtctgctctgcacatggccttttccgaccagcagtacctccacagaccctacktttatgctgtgtggtatgatgatgcaagtcgccatgcagcttggtcttcaccggccttcgcacactcaggactttagcaagtttacagtggagctgattgaggaggaactaagggacaaagtgaggacatgggcttcggtct

SEQ ID NO: 75 - DNA, 349 bp, Aspergillus versicolor
cgcccttgctggcaaccagagcccctctgcttccgatgttaaggaggttctctcctccgtcggtgttgacgctgactctgagcgcctcgagaagctcatcgctgagctccagggcaaggacatcaacgaggttcgttttgacagaaccgcttgaaattcttggcagcagactaatgaaacattttctgcagttgatcgctgagggtaccactaagctcgcttccgttccctctggcggtgctggtgctgctgctgctcccgctgctggtggcgctgctgccgctgaggcccctgccgccgagaaggaggaggagaaggaggagtccgacgaggacatgggcttcggtct

SEQ ID NO: 76 - DNA, 302 bp, Candida lusitaniae
ktattgttggkcaacgctggtaacaccgccccatctgctgctgacgtcaagaaggtcttggaatccgtctctattgaggttgaggacgacaaggttgagaagttgttggctgaagttgaaggcaagaacgtcgaagagttgattgccgagggtaacgagaagttgtcttctgttccatctggtgctccagctgctgctggtgccgctgctgcttctggttctactgaggctgctgctgaagagccacaagaagaagagaaggaggagtctgacgacgacatgggtttcggtttattcgatta

SEQ ID NO: 77 - DNA, 286 bp, Candida famata
cttctccatccgcttctgacatcagtagtttattagaaaccgttggtgctgaagctgacgaagctagaatcagtgctttattgaaggacttagaaggtaagcaagtcgctgacttaattgctgaaggtcaaaccaagttggcttccgttccaactggtggtgctggtgctgctgctggtggtgccgctgctgcttctggtgatgccggtgcagctgctgctgaagaagaaaaggaagaagaaaaggaagaatccgacgatgacatgggtttcggtttattcgatta

SEQ ID NO: 78 - DNA, 282 bp, Candida famata
cttctccatcagcctctgacgtcagtgctttattagaaaccgttggtgctgaagttgaccaaggtagagttagtgctttattgaaggacttagaaggtaagcaagttgccgacttaattgctgaaggtcaaaccaagttagcttctgtcccaaccggtggtgctgcttctgctggtggtgctgccgctgcttctggtgctgccggtgcagctgctgttgaagaagaaaaggaagaagaaaaggaagaatccgatgaagatatgggtttcggtttattcgatt

SEQ ID NO: 79 - DNA, 286 bp, Candida famata
cttctccatcagcctctgacgtcagtgctttattagaaaccgttggtgctgaagttgaccaaggtagagtcagtgctttattgaaggacttagaaggtaagcaagtcgccgacttaattgctgaaggtcaaaccaagttagcttctgtcccaactggtggtgctgcttctgctggtggtgcttccgctgctgcttctggtgatgccggtgcagctgctgctgaagaagaaaaggaagaagaaaaggaagaatccgatgaagatatgggtttcggtttattcgatta

SEQ ID NO: 80 - DNA, 298 bp, Candida haemulonii
tttcggtttattcgattaacacttccccagctgcctctgacatcaagaaggtgttggagtctgtctccatcgaggttgaggacgacaaggtcgagaagttgttggctgaggtcgagggcaagaacgccgaggagttgattgccgagggtaacgagaagttgtcttctgtcccaactggtgctccagctggtggtgctgccgctgctggtggtgctgctccagaggctgctgctgagaaggaagaggaggccgctgccgaggagtctgacgacgacatgggtttcggtttattcgatta

SEQ ID NO: 81 - DNA, 302 bp, Candida haemulonii
ttattgttggtcaacgccggtaacacttccccagctgcctctgacatcaagaaggtgttggagtctgtctccatcgaggttgaggacgacaaggtcgagaagttgttggctgaggtcgagggcaagaacgccgaggagttgattgccgagggtaacgagaagttgtcttctgtcccaactggtgctccagctggtggtgctgccgctgctggtggtgctgctccagaggctgctgctgagaaggaagaggaggccgctgccgaggagtctgacgacgacatgggtttcggtttattcgatta

SEQ ID NO: 82 - DNA, 280 bp, Candida pulcherrima
acacctctccatccgccgccgatgtcaagaaggtcttggagtccgtttccatcgaggttgaggaggacaaggtcgagaagttgctcgctgaggtcgagggcaagagcgtcgaggacttgatcgctgagggtaacgagaagttgtcttctgtcccaactggtggcccagccgccggtggtgccgctgccgctgctggtggtgacgccgctcctgccgaggaggccgctgaggaggccgccgaggagtctgacgacgacatgggtttcggtttattcgatta

SEQ ID NO: 83 - DNA, 271 bp, Candida pulcherrima
catccgccgccgatgtcaagraggtcttggagtccgtttccatcgaggttgaggaggacaagatcgagaagttgctcgctgaggtcgagggcaagagcgtcgaggacttgatcgctgagggtaacgagaagttgtcttctgtcccaactggtggcccagccgccggtggtgccgctgccgctgccggtggtgacgccgctcctgccgaggaggccgctgaggaggccgccgaggagtctgacgacgacatgggtttcggtttattcgatta

SEQ ID NO: 84 - DNA, 272 bp, Candida utilis
accgccgacaagatcacctccgtcttggagtctgtcggtattgaggttgaggagtcccaagtcaccgagttgatctctgcccttgagggtaagtccgttgaggagctcattgctgaaggtaacgagaagttggcttctgttccaaccggtggtgctggtgctgctccagctgccggtgccggtgctgctgatgctgatgctccagctgaggctgctgaggaggctgctgaggaggagtctgacgatgacatgggtttcggtttattcgatta

SEQ ID NO: 85 - DNA, 11 bp, Artificial Sequence
  Primer
gaggagcgcct

SEQ ID NO: 86 - DNA, 12 bp, Artificial Sequence
  Primer
gaggagcgcctc

SEQ ID NO: 87 - DNA, 13 bp, Artificial Sequence
  Primer
ggaggagcgcctc

SEQ ID NO: 88 - DNA, 13 bp, Artificial Sequence
  Primer
tgaggagcgcctc

SEQ ID NO: 89 - DNA, 14 bp, Artificial Sequence
  Primer
ccggagggaacgga

SEQ ID NO: 90 - DNA, 13 bp, Artificial Sequence
  Primer
ccggagggaacgg

SEQ ID NO: 91 - DNA, 18 bp, Artificial Sequence
  Primer
actacagctcgaagatta

SEQ ID NO: 92 - DNA, 18 bp, Artificial Sequence
  Primer
acgttgaatgattgagac

SEQ ID NO: 93 - DNA, 19 bp, Artificial Sequence
  Primer
ttgcgattacaagatggaa

SEQ ID NO: 94 - DNA, 19 bp, Artificial Sequence
  Primer
cttcggactactgcgatga

SEQ ID NO: 95 - DNA, 19 bp, Artificial Sequence
  Primer
acctctccatcagcttctg

SEQ ID NO: 96 - DNA, 22 bp, Artificial Sequence
  Primer
acctctgtcttatcatctgtcg

SEQ ID NO: 97 - DNA, 22 bp, Artificial Sequence
  Primer
aagaaggttatcgaatctgttg

SEQ ID NO: 98 - DNA, 22 bp, Artificial Sequence
  Primer
tccgctttattggaacaagttg

SEQ ID NO: 99 - DNA, 22 bp, Artificial Sequence
  Primer
tcctcattgttggaatccgttg

SEQ ID NO: 100 - DNA, 18 bp, Artificial Sequence
  Primer
tcagcaatcaattcttgc

SEQ ID NO: 101 - DNA, 18 bp, Artificial Sequence
  Primer
ctcttcgacggacttacc

SEQ ID NO: 102 - DNA, 18 bp, Artificial Sequence
  Primer
ttcgtccaaggacttacc

SEQ ID NO: 103 - DNA, 18 bp, Artificial Sequence
  Primer
ttcttgcaagtctttacc

SEQ ID NO: 104 - DNA, 18 bp, Artificial Sequence
  Primer
ctcgttgatgtctttacc

SEQ ID NO: 105 - DNA, 22 bp, Artificial Sequence
  Primer
accgctttattggaatccgttg

SEQ ID NO: 106 - DNA, 25 bp, Artificial Sequence
  Primer
atccgacaagttagacaagttaatc

SEQ ID NO: 107 - DNA, 23 bp, Artificial Sequence
  Primer
agaatcaacgaattgttgtctgc

SEQ ID NO: 108 - DNA, 26 bp, Artificial Sequence
  Primer
atcttccaaattagacttattgttga

SEQ ID NO: 109 - DNA, 27 bp, Artificial Sequence
  Primer
gaagaatcaagattatctaccttgttg

SEQ ID NO: 110 - DNA, 380 bp, Aspergillus fumigatus; identical to SEQ ID NO: 36